(19) United States
(12) Patent Application Publication
(10) Pub. No.: US 2025/0273343 A1
(43) Pub. Date: Aug. 28, 2025

(54) PREDICTIVE AND PRESCRIPTIVE ANALYTICS TO IDENTIFY PATIENT AND PREVENT FROM DEVELOPING OPIOID USE DISORDER

(71) Applicant: Stellar IT Solutions, Inc., Rockville, MD (US)

(72) Inventor: Dipak Thakker, Rockville, MD (US)

(21) Appl. No.: 19/170,547

(22) Filed: Apr. 4, 2025

Related U.S. Application Data
(60) Provisional application No. 63/549,703, filed on Feb. 5, 2024.

(51) Int. Cl. G16H 50/30; G16H 10/60; G16H 20/10

(52) U.S. Cl. CPC G16H 50/30; G16H 10/60; G16H 20/10

(57) ABSTRACT

Embodiments relate to a system comprising a processor that is configured to: receive input data of a patient, wherein the input data comprises one or more of a patient data, a prescription data, a drug data, a dispenser data, and a prescriber data; derive one or more attribute variables, based on the input data; predict using a predictive analytics, a risk score based on the attribute variables, wherein the risk score is a probability of the patient developing an opioid use disorder; and determine a subset of the attribute variables according to a percentage contribution to the risk score; provide using a prescriptive analytics module, a treatment recommendation based on the risk score and the attribute variables, and wherein the system is configured to identify and/or prevent opioid use disorder in the patient being treated for pain with a prescription drug, and wherein the prescription drug is an opioid.

### CROSS REFERENCE TO RELATED APPLICATIONS

This application claims the benefit under 35 U.S.C § 119 of U.S. Provisional Application No. 63/549,703, filed on Feb. 5, 2024, which is hereby incorporated by reference in its entirety.

### TECHNICAL FIELD

The present disclosure relates to identifying patients with opioid use disorder (OUD). More specifically, the present disclosure relates to systems and methods to identify patients developing opioid use disorder using explainable artificial intelligence models and further preventing the OUD through intervention.

### BACKGROUND

The sources supporting certain data and statements presented in this section are listed in the references section.

Opioid Use Disorder (OUD) has reached crisis levels in the US, affecting over 2 million Americans and killing 75,000 last year (2024). Over the last decade, OUD deaths have more than doubled and in multiple years increased by over 20% (max 28.5% from 2019 to 2020). From 2002-2017, the total number of fatalities involving opioids increased by 4.1-fold. In response, the Centers for Disease Control and Prevention (CDC) issued guidelines for opioid prescribing practices in 2016 which lowered prescription numbers by 9% in the first two years, but the duration and dosage significantly increased. While fatality statistics include both prescribed opioids and illicit drugs, 75% of OUD patients reported that their first opioid use was through a prescription.

A study examining opioid deaths within the last two years found that over 75,000 Americans died from opioid overdoses, with over two-thirds involving prescription opioids. 40% of deaths stemming from opioids can be traced back to the legal prescription of opioids from clinicians. Despite efforts to combat the negative effects of opioid prescription, the amount (in mg) of opioids prescribed is ˜3× higher than it was in 1999. Hospitalizations for prescription opioid overdoses have exceeded heroin-related hospitalizations for years, though illicit production and consumption of fentanyl has emerged as the predominant cause of hospitalizations. Again, the majority of these hospitalizations, even if resulting from illicit consumption, involved patients that initially started using opioids via a prescription from their doctor.

Therefore, there is a need for a system, a method, and/or a product or tool to aid physicians in the prediction and identification of patients at risk of developing opioid use disorder or substance abuse for prescription drugs.

### SUMMARY

The following presents a summary to provide a basic understanding of one or more embodiments described herein. This summary is not intended to identify key or critical elements or delineate any scope of the different embodiments and/or any scope of the claims. The sole purpose of the summary is to present some concepts in a simplified form as a prelude to the more detailed description presented herein.

According to an embodiment, it is a system comprising, a processor storing instructions in a non-transitory memory that, when executed, cause the processor to: receive, input data of a patient, wherein the input data comprises one or more of a first patient data, a first prescription data, a first drug data, a first dispenser data, and a first prescriber data; derive, one or more attribute variables, based on the input data, wherein the attribute variables comprise one or more of a second patient data, a second prescription data, a second drug data, a second dispenser data, and a second prescriber data; predict, using a predictive analytics module comprising a first artificial intelligence and machine learning model, a risk score for the patient based on the attribute variables, wherein the risk score is a probability of the patient developing an opioid use disorder, wherein the first artificial intelligence and machine learning model that provides explainable outcome and is pre-trained on a training database; and determine, a list of the attribute variables that contribute to the risk score, wherein the list comprises a percentage contribution of each of the attribute variables in the list to the risk score, wherein the list comprises a subset of the attribute variables; provide, using a prescriptive analytics module comprising a second artificial intelligence and machine learning model, a treatment recommendation based on the risk score and the attribute variables, wherein the second artificial intelligence and machine learning model is pre-trained on the training database, wherein the treatment recommendation comprises one or more of a dosage, an alternate medication, a craving-suppression medication, a behavioral intervention, a therapy, a withdrawal reduction treatment, and educational materials to overcome the opioid use disorder; and wherein the system is configured to one or more of identifying and preventing the opioid use disorder in the patient, wherein the patient is being treated for pain with a drug via prescription, and wherein the drug is an opioid.

According to an embodiment of the system, the input data is considered from one or more databases, wherein the databases comprise Prescription Drug Monitoring Program (PDMP) and Electronic Health Record (EHR).

According to an embodiment of the system, the first patient data comprises at least one or more of patient identification details, patient date of birth, and patient location.

According to an embodiment of the system, the first prescription data comprises at least one or more of prescription filled data, prescription date, and prescription number.

According to an embodiment of the system, the first drug data comprises at least one or more of drug name, drug strength, drug form, drug quantity, and number of days of supply.

According to an embodiment of the system, the first dispenser data comprises one or more of dispenser identification details and dispenser location.

According to an embodiment of the system, the first prescriber data comprises one or more of prescriber identification details and prescriber location.

According to an embodiment of the system, the second patient data comprises one or more of patient unique identification, patient age, and patient location.

According to an embodiment of the system, the second prescription data comprises one or more of number of prescriptions in a time period, days since last prescription, average number of prescriptions per period, and average duration between prescriptions.

According to an embodiment of the system, the second drug data comprises one or more of average drug quantity, highest drug quantity, average days of supply, highest days of supply, highest drug strength, lowest drug strength as per the first prescription data and the first drug data.

According to an embodiment of the system, the second dispenser data comprises one or more of dispenser identification details, dispenser location, highest number of prescriptions filled by a dispenser, identification details of most frequently used dispenser for filling the drug.

According to an embodiment of the system, the second prescriber data comprises one or more of prescriber identification details, prescriber location, highest number of prescriptions prescribed, and most frequent prescriber identification details.

According to an embodiment of the system, the first artificial intelligence and machine learning model comprises a logistic regression model configured to explain why a patient is at risk.

According to an embodiment of the system, the logistic regression model applies a linear and weighted contribution of the input data; and wherein the logistic regression model comprises a multivariate logistic regression.

According to an embodiment of the system, the risk score is a value between 0 and 1.

According to an embodiment of the system, the second artificial intelligence and machine learning model comprises a clustering model.

According to an embodiment of the system, the clustering model comprises one of recursive partitioning and random forest clustering.

According to an embodiment of the system, the clustering model groups the patient with similar underlying attributes into a cluster of the plurality of patients from training input data.

According to an embodiment of the system, the training dataset comprises the training input data from a plurality of patients.

According to an embodiment of the system, the training input data is preprocessed by cleaning and normalizing the training input data.

According to an embodiment of the system, the system is configured for use by a physician as a decision support system for prescribing one or more of the prescription drug and the treatment recommendation.

According to an embodiment, it is a method comprising, receiving, input data of a patient, wherein the input data comprises one or more of a first patient data, a first prescription data, a first drug data, a first dispenser data, and a first prescriber data; deriving, one or more attribute variables, based on the input data, wherein the attribute variables comprise one or more of a second patient data, a second prescription data, a second drug data, a second dispenser data, and a second prescriber data; predicting, using a predictive analytics module comprising a first artificial intelligence and machine learning model, a risk score for the patient based on the attribute variables, wherein the risk score is a probability of the patient developing an opioid use disorder, wherein the first artificial intelligence and machine learning model that provides explainable outcome and is pre-trained on a training database; and determining, a list of the attribute variables that contribute to the risk score, wherein the list comprises a percentage contribution of each of the attribute variables in the list to the risk score, wherein the list comprises a subset of the attribute variables; providing, using a prescriptive analytics module comprising a second artificial intelligence and machine learning model, a treatment recommendation based on the risk score and the attribute variables, wherein the second artificial intelligence and machine learning model is pre-trained on the training database, wherein the treatment recommendation comprises one or more of a dosage, an alternate medication, and craving-suppression medication, a behavioral intervention, a therapy, a withdrawal reduction treatment, and educational materials to overcome the opioid use disorder; and wherein the method is configured to one or more of identifying and preventing the opioid use disorder in the patient, wherein the patient is being treated for pain with a drug via prescription, and wherein the drug is an opioid.

According to an embodiment of the method, wherein the risk score along with the list of attributes is one or more of displayed on a display, stored to a database, and generates an alarm.

According to an embodiment of the method, the first artificial intelligence and machine learning model and the second artificial intelligence and machine learning model are retrained based on false positive and false negative results during a period of use of the first artificial intelligence and machine learning model and the second artificial intelligence and machine learning model.

According to an embodiment, it is a non-transitory computer-readable medium having stored thereon instructions executable by a computer system to perform operations comprising receiving, input data of a patient, wherein the input data comprises one or more of a first patient data, a first prescription data, a first drug data, a first dispenser data, and a first prescriber data; deriving, one or more attribute variables, based on the input data, wherein the attribute variables comprise one or more of a second patient data, a second prescription data, a second drug data, a second dispenser data, and a second prescriber data; predicting, using a predictive analytics module comprising a first artificial intelligence and machine learning model, a risk score for the patient based on the attribute variables, wherein the risk score is a probability of the patient developing an opioid use disorder, wherein the first artificial intelligence and machine learning model that provides explainable outcome and is pre-trained on a training database; and determining, a list of the attribute variables that contribute to the risk score, wherein the list comprises a percentage contribution of each of the attribute variables in the list to the risk score, wherein the list comprises a subset of the attribute variables; providing, using a prescriptive analytics module comprising a second artificial intelligence and machine learning model, a treatment recommendation based on the risk score and the attribute variables, wherein the second artificial intelligence and machine learning model is pre-trained on the training database, wherein the treatment recommendation comprises one or more of a dosage, an alternate medication, and craving-suppression medication, a behavioral intervention, a therapy, a withdrawal reduction treatment, and educational materials to overcome the opioid use disorder; and wherein the operations are configured to one or more of identifying and preventing the opioid use disorder in the patient, wherein the patient is being treated for pain with a drug via prescription, and wherein the drug is an opioid.

According to an embodiment, it is a system comprising a processor storing instructions in a non-transitory memory that, when executed, cause the processor to acquire patient data related to patients; preprocess the patient data to obtain preprocessed patient data; train, a first machine learning model with the preprocessed patient data, wherein the first machine learning model is configured to: analyze one or more features from the preprocessed patient data of the patient; predict a risk score of the patient at risk of developing opioid use disorder; and compute prescriptive analytics to provide a treatment recommendation based on the predicted risk score; and wherein the trained first machine learning model is configured to identify patients and prevent developing opioid use disorder.

According to an embodiment of the system, the processor is configured to apply logistic distribution to a linear, weighted combination of the one or more features to produce the risk score between 0 and 1.

According to an embodiment of the system, the preprocessed patient data comprises at least one of structured data, semi-structured data, and unstructured data.

According to an embodiment of the system, the preprocessed patient data is stored in a database.

According to an embodiment of the system, the patient data comprises at least one of a textual data, a numerical data, a graphical representation, a chart, and a table.

According to an embodiment of the system, the first machine learning model is further configured to: cleanse and filter the preprocessed patient data based on at least one of an input from a user, and a predefined rule.

According to an embodiment of the system, the first machine learning model is configured to perform one or more of a normalization, a standardization, and a stratification of the preprocessed patient data.

According to an embodiment of the system, the system is further configured to: customize the first machine learning model by at least one of manipulating the preprocessed patient data to obtain manipulated preprocessed patient data, adding, modifying, removing at least one node of the first machine learning model, and train the first machine learning model using the manipulated preprocessed patient data.

According to an embodiment of the system, the system is further configured to enable a user to interact with a server through a user interface, provided via a device associated with the user, and at least one of build, train, retrain, replicate, compare, and share the first machine learning model.

According to an embodiment of the system, the system is configured to display the risk score.

According to an embodiment of the system, the first machine learning model is calibrated and selected from one or more artificial intelligence (AI) models for computing predictive and prescriptive analytics by evaluating AI model candidates against a set of performance criteria.

According to an embodiment of the system, the set of performance criteria comprise prediction accuracy, computational efficiency, and adaptability to diverse preprocessed patient data.

According to an embodiment of the system, the first machine learning model is configured to learn using labeled data using a supervised learning model, wherein the supervised learning model comprises logic using at least one of a decision tree, a logistic regression, a support vector machine, a k-nearest neighbors, a Naïve Bayes, a random forest, a linear regression, a polynomial regression, and a support vector machine for regression.

According to an embodiment of the system, the first machine learning model has a feedback loop, wherein an output from a previous step is fed back to the model in real-time to improve performance and accuracy of the output of a next step.

According to an embodiment of the system, the first machine learning model is a self-learning model.

According to an embodiment of the system, the first machine learning model comprises a feedback loop, wherein the learning is further reinforced with a reward for each true positive of an output of the system.

According to an embodiment of the system, the processor is further configured to assign a weight to the one or more features based on a first pattern of outcomes of historical patient data.

According to an embodiment of the system, the processor is further configured to modify the weight of the one or more features based on a second pattern of outcomes of training patient data.

### BRIEF DESCRIPTION OF THE FIGURES

These and other aspects of the present disclosure will now be described in more detail, with reference to the appended drawings showing exemplary embodiments in the present disclosure, in which:

FIG. 1 illustrates a system for identification and prevention of Opioid Use Disorder (OUD) in patients using OUD Risk Tool, according to one or more embodiments.

FIG. 2A illustrates various modules of the system for opioid use disorder prediction and prevention according to an embodiment.

FIG. 2B is an illustration of unsupervised machine learning algorithms grouping patient data into clusters according to an embodiment.

FIG. 3 illustrates a predictive analytics module according to an embodiment.

FIG. 4 illustrates predictive data/attributes and the source from which the data is collected according to an embodiment.

FIG. 5 shows raw data taken from the Prescription Drug Monitoring Program (PDMP) database and derived attributes according to an embodiment.

FIG. 6A illustrates Prescription Drug Monitoring Program (PDMP) Data attributes used in predictive analytics and prescriptive analytics according to an embodiment.

FIG. 6B illustrates a table showing all the prescription data over the last 24 months summarized or aggregated to calculate attribute values for each patient according to an embodiment.

FIG. 7 illustrates Decision Support System for providing patient-specific prediction and treatment prescription, according to an embodiment.

FIG. 8 shows the process steps involved in development and implementation of the proposed solution according to an embodiment.

FIG. 9A illustrates Opioid Use Disorder (OUD) Architecture for Prediction Analytics according to an embodiment.

FIG. 9B illustrates the OUD predictive analytics and different stages involved in predictive analytics module development and deployment according to an embodiment.

FIG. 9C illustrates an output from the interactive dashboard from the predictive analytics module according to an embodiment.

FIG. 9D shows a list of some of the patients with their individual risk score as per predictive analysis according to an embodiment.

FIG. 9E illustrates the percentage of contribution of attribute variables to the risk score for the patient according to an embodiment.

FIG. 9F shows examples of derivation of descriptive risk scores according to an embodiment.

FIG. 10 illustrates methodology for algorithm development according to an embodiment.

FIG. 11 illustrates data/attributes used in predictive analytics for retention in care of Primary care in People With Human Immunodeficiency Virus (PWH) according to an embodiment.

FIG. 12A shows a structure of the neural network/machine learning model with a feedback loop.

FIG. 12B shows a structure of the neural network/machine learning model with reinforcement learning.

FIG. 13 illustrates a flow chart describing a method implemented in the opioid risk tool according to an embodiment.

FIG. 14 illustrates a block diagram of the system implementing the method according to an embodiment.

FIG. 15 illustrates a block diagram of the method executed by the non-transitory computer-readable medium according to an embodiment.

FIG. 16A illustrates a block diagram of the cyber security module in view of the system and server according to an embodiment.

FIG. 16B illustrates a block diagram of the cyber security module according to an embodiment.

FIG. 16C illustrates another block diagram of the cyber security module according to an embodiment.

Other features of the present embodiments will be apparent from the accompanying drawings and from the detailed description that follows.

### DETAILED DESCRIPTION

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments in the present disclosure. The same reference numeral in different figures denotes the same elements.

Although the detailed description herein contains many specifics for the purpose of illustration, a person of ordinary skill in the art will appreciate that many variations and alterations to the details are considered to be included herein.

Accordingly, the embodiments herein are without any loss of generality to, and without imposing limitations upon, any claims set forth. The terminology used herein is for the purpose of describing particular embodiments only and is not limiting. Unless defined otherwise, all technical and scientific terms used herein have the same meaning as commonly understood by one with ordinary skill in the art in the field of this disclosure. The following terms and phrases, unless otherwise indicated, shall be understood to have the following meanings.

As used herein, the articles “a” and “an” used herein refer to one or to more than one (i.e., to at least one) of the grammatical object of the article. By way of example, “an element” means one element or more than one element. Moreover, usage of articles “a” and “an” in the subject specification and annexed drawings construe to mean “one or more” unless specified otherwise or clear from context to mean a singular form.

As used herein, the terms “example” and/or “exemplary” mean serving as an example, instance, or illustration. For the avoidance of doubt, such examples do not limit the herein described subject matter. In addition, any aspect or design described herein as an “example” and/or “exemplary” is not necessarily preferred or advantageous over other aspects or designs, nor does it preclude equivalent exemplary structures and techniques known to those of ordinary skill in the art.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

No element, act, or instruction used herein should be construed as critical or essential unless explicitly described as such. Also, as used herein, the articles “a” and “an” are intended to include items and may be used interchangeably with “one or more.” Furthermore, as used herein, the term “set” is intended to include items (e.g., related items, unrelated items, a combination of related items, and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

As used herein, two or more elements or modules are “integral” or “integrated” if they operate functionally together. Two or more elements are “non-integral” if each element can operate functionally independently.

As defined herein, “real-time” can, in some embodiments, be defined with respect to operations carried out as soon as practically possible upon occurrence of a triggering event. A triggering event can include receipt of data necessary to execute a task or to otherwise process information. Because of delays inherent in transmission and/or in computing speeds, the term “real-time” encompasses operations that occur in “near” real-time or somewhat delayed from a triggering event. In a number of embodiments, “real-time” can mean real-time less a time delay for processing (e.g., determining) and/or transmitting data. The particular time delay can vary depending on the type and/or amount of the data, the processing speeds of the hardware, the transmission capability of the communication hardware, the transmission distance, etc. However, in many embodiments, the time delay can be less than approximately one second, two seconds, five seconds, or ten seconds.

As used herein, the term “approximately” can mean within a specified or unspecified range of the specified or unspecified stated value. In some embodiments, “approximately” can mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

As used herein the term “component” refers to a distinct and identifiable part, element, or unit within a larger system, structure, or entity. It is a building block that serves a specific function or purpose within a more complex whole. Components are often designed to be modular and interchangeable, allowing them to be combined or replaced in various configurations to create or modify systems. Components may be a combination of mechanical, electrical, hardware, firmware, software, and/or other engineering elements.

Digital electronic circuitry, or computer software, firmware, or hardware, including the structures disclosed in this specification and their structural equivalents, or in combinations of one or more of them may realize the implementations and all of the functional operations described in this specification. Implementations may be as one or more computer program products, i.e., one or more modules of computer program instructions encoded on a computer-readable medium for execution by, or to control the operation of, data processing apparatus. The computer-readable medium may be a machine-readable storage device, a machine-readable storage substrate, a memory device, a composition of matter affecting a machine-readable propagated signal, or a combination of one or more of them. The term “computing system” encompasses all apparatus, devices, and machines for processing data, including by way of example, a programmable processor, a computer, or multiple processors or computers. The apparatus may include, in addition to hardware, code that creates an execution environment for the computer program in question, e.g., code that constitutes processor firmware, a protocol stack, a database management system, an operating system, or a combination of one or more of them. A propagated signal is an artificially generated signal (e.g., a machine-generated electrical, optical, or electromagnetic signal) that encodes information for transmission to a suitable receiver apparatus.

The actual specialized control hardware or software code used to implement these systems and/or methods is not limiting to the implementations. Thus, any software and any hardware can implement the systems and/or methods based on the description herein without reference to specific software code.

A computer program (also known as a program, software, software application, script, or code) is written in any appropriate form of programming language, including compiled or interpreted languages. Any appropriate form, including a stand-alone program or a module, component, subroutine, or other unit suitable for use in a computing environment may deploy it. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may execute on one computer or on multiple computers that are located at one site or distributed across multiple sites and interconnected by a communication network.

One or more programmable processors, executing one or more computer programs to perform functions by operating on input data and generating output, perform the processes and logic flows described in this specification. The processes and logic flows may also be performed by, and apparatus may also be implemented as special purpose logic circuitry, for example, without limitation, a Field Programmable Gate Array (FPGA), an Application Specific Integrated Circuit (ASIC), Application Specific Standard Products (ASSPs), System-On-a-Chip (SOC) systems, Complex Programmable Logic Devices (CPLDs), etc.

Processors suitable for the execution of a computer program include, by way of example, both general and special purpose microprocessors, and any one or more processors of any appropriate kind of digital computer. A processor receives instructions and data from a read-only memory or a random-access memory or both. Elements of a computer can include a processor for performing instructions and one or more memory devices for storing instructions and data. A computer includes, or is operatively coupled to receive data, transfer data or both, to/from one or more mass storage devices for storing data e.g., magnetic disks, magneto optical disks, optical disks, or solid-state disks. However, a computer need not have such devices. Moreover, another device, e.g., a mobile telephone, a personal digital assistant (PDA), a mobile audio player, a Global Positioning System (GPS) receiver, etc. may embed a computer. Computer-readable media suitable for storing computer program instructions and data include all forms of non-volatile memory, media and memory devices, including, by way of example, semiconductor memory devices (e.g., Erasable Programmable Read-Only Memory (EPROM), Electronically Erasable Programmable Read-Only Memory (EEPROM), and flash memory devices), magnetic disks (e.g., internal hard disks or removable disks), magneto optical disks (e.g. Compact Disk Read-Only Memory (CD-ROM) disks, Digital Versatile Disk-Read-Only Memory (DVD-ROM) disks) and solid-state disks. Special purpose logic circuitry may supplement or incorporate the processor and the memory.

To provide for interaction with a user, a computer may have a display device, e.g., a Cathode Ray Tube (CRT) or Liquid Crystal Display (LCD) monitor, for displaying information to the user, and a keyboard and a pointing device, e.g., a mouse or a trackball, by which the user may provide input to the computer. Other kinds of devices provide for interaction with a user as well. For example, feedback to the user may be any appropriate form of sensory feedback, e.g., visual feedback, auditory feedback, or tactile feedback; and a computer may receive input from the user in any appropriate form, including acoustic, speech, or tactile input.

A computing system that includes a back-end component, e.g., a data server, or that includes a middleware component, e.g., an application server, or that includes a front-end component, e.g., a client computer having a graphical user interface or a Web browser through which a user may interact with an implementation, or any appropriate combination of one or more such back-end, middleware, or front-end components, may realize implementations described herein. Any appropriate form or medium of digital data communication, e.g., a communication network, may interconnect the components of the system. Examples of communication networks include a Local Area Network (LAN) and a Wide Area Network (WAN), e.g., Intranet and Internet.

The computing system may include clients and servers. A client and server are remote from each other and typically interact through a communication network. The relationship of the client and server arises by virtue of computer programs running on the respective computers and having a client-server relationship with each other.

Embodiments in the present disclosure may comprise or utilize a special purpose or general-purpose computer including computer hardware. Embodiments within the scope of the present disclosure may also include physical and other computer-readable media for carrying or storing computer-executable instructions and/or data structures. Such computer-readable media can be any media accessible by a general purpose or special purpose computer system. Computer-readable media that store computer-executable instructions are physical storage media. Computer-readable media that carry computer-executable instructions are transmission media. Thus, by way of example and not limitation, embodiments in the disclosure can comprise at least two distinct kinds of computer-readable media: physical computer-readable storage media and transmission computer-readable media.

Although the present embodiments described herein are with reference to specific example embodiments it will be evident that various modifications and changes may be made to these embodiments without departing from the broader spirit and scope of the various embodiments. For example, hardware circuitry (e.g., Complementary Metal Oxide Semiconductor (CMOS) based logic circuitry), firmware, software (e.g., embodied in a non-transitory machine-readable medium), or any combination of hardware, firmware, and software may enable and operate the various devices, units, and modules described herein. For example, transistors, logic gates, and electrical circuits (e.g., Application Specific Integrated Circuit (ASIC) and/or Digital Signal Processor (DSP) circuit) may embody the various electrical structures and methods.

In addition, a non-transitory machine-readable medium and/or a system may embody the various operations, processes, and methods disclosed herein. Accordingly, the specifications and drawings are illustrative rather than restrictive.

Physical computer-readable storage media includes RAM, ROM, EEPROM, CD-ROM or other optical disk storage (such as CDs, DVDs, etc.), magnetic disk storage or other magnetic storage devices, solid-state disks or any other medium. They store desired program code in the form of computer-executable instructions or data structures which can be accessed by a general purpose or special purpose computer.

As used herein, the term “network” refers to one or more data links that enable the transport of electronic data between computer systems and/or modules and/or other electronic devices. When a network or another communications connection (either hardwired, wireless, or a combination of hardwired or wireless) transfers or provides information to a computer, the computer properly views the connection as a transmission medium. A general purpose or special purpose computer can access transmission media that can include a network and/or data links which carry desired program code in the form of computer-executable instructions or data structures. The scope of computer-readable media includes combinations of the above, that enable the transport of electronic data between computer systems and/or modules and/or other electronic devices. The term network may include the Internet, a local area network, a wide area network, or combinations thereof. The network may include one or more networks or communication systems, such as the Internet, the telephone system, satellite networks, cable television networks, and various other private and public networks. In addition, the connections may include wired connections (such as wires, cables, fiber optic lines, etc.), wireless connections, or combinations thereof. Furthermore, although not shown, other computers, systems, devices, and networks may also be connected to the network. Network refers to any set of devices or subsystems connected by links joining (directly or indirectly) a set of terminal nodes sharing resources located on or provided by network nodes. The computers use common communication protocols over digital interconnections to communicate with each other. For example, subsystems may comprise the cloud. Cloud refers to servers that are accessed over the Internet, and the software and databases that run on those servers.

Further, upon reaching various computer system components, program code in the form of computer-executable instructions or data structures can be transferred automatically from transmission computer-readable media to physical computer-readable storage media (or vice versa). For example, computer-executable instructions or data structures received over a network or data link can be buffered in RAM within a Network Interface Controller (NIC), and then eventually transferred to computer system RAM and/or to less volatile computer-readable physical storage media at a computer system. Thus, computer system components that also (or even primarily) utilize transmission media may include computer-readable physical storage media.

Computer-executable instructions comprise, for example, instructions and data which cause a general-purpose computer, special purpose computer, or special purpose processing device to perform a certain function or group of functions. The computer-executable instructions may be, for example, binary, intermediate format instructions such as assembly language, or even source code. Although the subject matter herein described is in a language specific to structural features and/or methodological acts, the described features or acts described do not limit the subject matter defined in the claims. Rather, the herein described features and acts are example forms of implementing the claims.

While this specification contains many specifics, these do not construe as limitations on the scope of the disclosure or of the claims, but as descriptions of features specific to particular implementations. A single implementation may implement certain features described in this specification in the context of separate implementations. Conversely, multiple implementations separately or in any suitable sub-combination may implement various features described herein in the context of a single implementation. Moreover, although features described herein as acting in certain combinations and even initially claimed as such, one or more features from a claimed combination may in some cases be excised from the combination, and the claimed combination may be directed to a sub-combination or variation of a sub-combination.

Similarly, while operations depicted herein in the drawings are in a particular order to achieve desired results, this should not be understood as requiring that such operations be performed in the particular order shown, or in sequential order, or that all illustrated operations be performed, to achieve desirable results. In certain circumstances, multitasking and parallel processing may be advantageous. Moreover, the separation of various system components in the implementations should not be understood as requiring such separation in all implementations, and it should be understood that the described program components and systems may be integrated together in a single software product or packaged into multiple software products.

Even though particular combinations of features are recited in the claims and/or disclosed in the specification, these combinations are not intended to limit the disclosure of possible implementations. Other implementations are within the scope of the claims. For example, the actions recited in the claims may be performed in a different order and still achieve desirable results. In fact, many of these features may be combined in ways not specifically recited in the claims and/or disclosed in the specification. Although each dependent claim may directly depend on only one claim, the disclosure of possible implementations includes each dependent claim in combination with every other claim in the claim set.

Further, a computer system including one or more processors and computer-readable media such as computer memory may practice the methods. In particular, one or more processors execute computer-executable instructions, stored in the computer memory, to perform various functions such as the acts recited in the embodiments.

Those skilled in the art will appreciate that the disclosure may be practiced in network computing environments with many types of computer system configurations including personal computers, desktop computers, laptop computers, message processors, hand-held devices, multi-processor systems, microprocessor-based or programmable consumer electronics, network PCs, mini-computers, mainframe computers, mobile telephones, PDAs, pagers, routers, switches, etc. Distributed system environments where local and remote computer systems, which are linked (either by hardwired data links, wireless data links, or by a combination of hardwired and wireless data links) through a network, both perform tasks may also practice the disclosure. In a distributed system environment, program modules may be located in both local and remote memory storage devices.

As used herein, the term “unauthorized access” is when someone gains access to a website, program, server, service, or other system using someone else's account or other methods. For example, if someone kept guessing a password or username for an account that was not theirs until they gained access, it is considered unauthorized access.

As used herein, the term “IoT” stands for Internet of Things which describes the network of physical objects “things” or objects embedded with sensors, software, and other technologies for the purpose of connecting and exchanging data with other devices and systems over the internet.

As used herein “machine learning” refers to algorithms that give a computer the ability to learn without explicit programming, including algorithms that learn from and make predictions about data. Machine learning techniques include, but are not limited to, support vector machine, artificial neural network (ANN) (also referred to herein as a “neural net”), deep learning neural network, logistic regression, discriminant analysis, random forest, linear regression, rule-based machine learning, Naive Bayes, nearest neighbor, decision tree, decision tree learning, and hidden Markov, etc. For clarity purposes, part of a machine learning process can use algorithms such as linear regression or logistic regression. However, using linear regression or another algorithm as part of a machine learning process is distinct from performing a statistical analysis such as regression with a spreadsheet program. The machine learning process can continually learn and adjust the classifier as new data becomes available and does not rely on explicit or rule-based programming. The ANN may be featured with a feedback loop to adjust the system output dynamically as it learns from the new data as it becomes available. In machine learning, backpropagation and feedback loops are used to train the Artificial Intelligence/Machine Learning (AI/ML) model, improving the model's accuracy and performance over time. Statistical modeling relies on finding relationships between variables (e.g., mathematical equations) to predict an outcome.

As used herein, the term “data mining” is a process used to turn raw data into useful information. It is the process of analyzing large datasets to uncover hidden patterns, relationships, and insights that can be useful for decision-making and prediction.

As used herein, the term “data acquisition” is the process of sampling signals that measure real world physical conditions and converting the resulting samples into digital numeric values that a computer manipulates. Data acquisition systems typically convert analog waveforms into digital values for processing. The components of data acquisition systems include sensors to convert physical parameters to electrical signals, signal conditioning circuitry to convert sensor signals into a form that can be converted to digital values, and analog-to-digital converters to convert conditioned sensor signals to digital values. Stand-alone data acquisition systems are often called data loggers.

As used herein, the term “dashboard” is a type of interface that visualizes particular Key Performance Indicators (KPIs) for a specific goal or process. It is based on data visualization and infographics.

As used herein, a “database” is a collection of organized information so that it can be easily accessed, managed, and updated. Computer databases typically contain aggregations of data records or files.

As used herein, the term “data set” (or “dataset”) is a collection of data. In the case of tabular data, a data set corresponds to one or more database tables, where every column of a table represents a particular variable, and each row corresponds to a given record of the data set in question. The data set lists values for each of the variables, such as height and weight of an object, for each member of the data set. Each value is known as a datum. Data sets can also consist of a collection of documents or files.

The term “communication module” or “communication unit” or “communication system” as used herein refers to a system which enables the information exchange between two points. The process of transmission and reception of information is called communication. The elements of communication include but are not limited to a transmitter of information, channel or medium of communication and a receiver of information.

The term “communication” as used herein refers to the transmission of information and/or data from one point to another. Communication may be by means of electromagnetic waves. Communication is also a flow of information from one point, known as the source, to another, the receiver. Communication comprises one of the following: transmitting data, instructions, information or a combination of data, instructions, and information. Communication happens between any two communication systems or communication units.

The terms “non-transitory computer-readable medium” and “computer-readable medium” include a single medium or multiple media such as a centralized or distributed database, and/or associated caches and servers that store one or more sets of instructions. Further, the terms “non-transitory computer-readable medium” and “computer-readable medium” include any tangible medium that is capable of storing, encoding, or carrying a set of instructions for execution by a processor that, for example, when executed, cause a system to perform any one or more of the methods or operations disclosed herein. As used herein, the term “computer-readable medium” is expressly defined to include any type of computer-readable storage device and/or storage disk and to exclude propagating signals.

The term “application server” refers to a server that hosts applications or software that delivers a business application through a communication protocol. An application server framework is a service layer model. It includes software components available to a software developer through an application programming interface. It is system software that resides between the operating system (OS) on one side, the external resources such as a database management system (DBMS), communications and Internet services on another side, and the users' applications on the third side.

The term “cyber security” as used herein refers to application of technologies, processes, and controls to protect systems, networks, programs, devices, and data from cyber-attacks.

The term “cyber security module” as used herein refers to a module comprising application of technologies, processes, and controls to protect systems, networks, programs, devices and data from cyber-attacks and threats. It aims to reduce the risk of cyber-attacks and protect against the unauthorized exploitation of systems, networks, and technologies. It includes, but is not limited to, critical infrastructure security, application security, network security, cloud security, Internet of Things (IoT) security.

The term “encrypt” used herein refers to securing digital data using one or more mathematical techniques, along with a password or “key” used to decrypt the information. It refers to converting information or data into a code, especially to prevent unauthorized access. It may also refer to concealing information or data by converting it into a code. It may also be referred to as cipher, code, encipher, encode. A simple example is representing alphabets with numbers-say, ‘A’ is ‘01’, ‘B’ is ‘02’, and so on. For example, a message like “HELLO” is encrypted as “0805121215,” and this value is transmitted over the network to the recipient(s).

The term “decrypt” used herein refers to the process of converting an encrypted message back to its original format. It is generally a reverse process of encryption. It decodes the encrypted information so that only an authorized user can decrypt the data because decryption requires a secret key or password. This term could be used to describe a method of unencrypting the data manually or unencrypting the data using the proper codes or keys.

The term “cyber security threat” used herein refers to any possible malicious attack that seeks to unlawfully access data, disrupt digital operations, or damage information. A malicious act includes but is not limited to damaging data, stealing data, or disrupting digital life in general. Cyber threats include, but are not limited to, malware, spyware, phishing attacks, ransomware, zero-day exploits, trojans, advanced persistent threats, wiper attacks, data manipulation, data destruction, rogue software, malvertising, unpatched software, computer viruses, man-in-the-middle attacks, data breaches, Denial of Service (DOS) attacks, and other attack vectors.

The term “hash value” used herein can be thought of as fingerprints for files. The contents of a file are processed through a cryptographic algorithm, and a unique numerical value, the hash value, is produced that identifies the contents of the file. If the contents are modified in any way, the value of the hash also changes significantly. Example algorithms used to produce hash values: the Message Digest-5 (MD5) algorithm and Secure Hash Algorithm-1 (SHA1).

The term “integrity check” as used herein refers to the checking for accuracy and consistency of system related files, data, etc. It may be performed using checking tools that can detect whether any critical system files have been changed, thus enabling the system administrator to look for unauthorized alteration of the system. For example, data integrity corresponds to the quality of data in the databases and to the level by which users examine data quality, integrity, and reliability. Data integrity checks verify that the data in the database is accurate, and functions as expected within a given application.

The term “alarm” as used herein refers to a trigger when a component in a system or the system fails or does not perform as expected. The system may enter an alarm state when a certain event occurs. An alarm indication signal is a visual signal to indicate the alarm state. For example, when a cyber security threat is detected, a system administrator may be alerted via sound alarm, a message, a glowing LED, a pop-up window, etc. Alarm indication signals may be reported downstream from a detecting device, to prevent adverse situations or cascading effects.

As used herein, the term “cryptographic protocol” is also known as security protocol or encryption protocol. It is an abstract or concrete protocol that performs a security-related function and applies cryptographic methods often as sequences of cryptographic primitives. A protocol describes how the algorithms should be used. A sufficiently detailed protocol includes details about data structures and representations, at which point it can be used to implement multiple, interoperable versions of a program. Cryptographic protocols are widely used for secure application-level data transport. A cryptographic protocol usually incorporates at least some of these aspects: key agreement or establishment, entity authentication, symmetric encryption, and message authentication material construction, secured application-level data transport, non-repudiation methods, secret sharing methods, and secure multi-party computation. Hashing algorithms may be used to verify the integrity of data. Secure Socket Layer (SSL) and Transport Layer Security (TLS), the successor to SSL, are cryptographic protocols that may be used by networking switches to secure data communication over a network.

The embodiments described herein can be directed to one or more of a system, a method, an apparatus, and/or a computer program product at any possible technical detail level of integration. The computer program product can include a computer-readable storage medium (or media) having computer-readable program instructions thereon for causing a processor to carry out aspects of the one or more embodiments described herein.

The flowcharts and block diagrams in the figures illustrate the architecture, functionality and/or operation of possible implementations of systems, computer-implementable methods and/or computer program products according to one or more embodiments described herein. In this regard, each block in the flowchart or block diagrams can represent a module, segment and/or portion of instructions, which comprises one or more executable instructions for implementing the specified logical function(s). In one or more alternative implementations, the functions noted in the blocks can occur out of the order noted in the Figures. For example, two blocks shown in succession can be executed substantially concurrently, and/or the blocks can sometimes be executed in the reverse order, depending upon the functionality involved. It will also be noted that each block of the block diagrams and/or flowchart illustration, and/or combinations of blocks in the block diagrams and/or flowchart illustration, can be implemented by special purpose hardware-based systems that can perform the specified functions and/or acts and/or carry out one or more combinations of special purpose hardware and/or computer instructions.

As used in this application, the terms “component,” “system,” “platform,” “interface,” and/or the like, can refer to and/or can include a computer-related entity or an entity related to an operational machine with one or more specific functionalities. The entities described herein can be either hardware, a combination of hardware and software, software, or software in execution. For example, a component can be, but is not limited to being, a process running on a processor, a processor, an object, an executable, a thread of execution, a program and/or a computer. By way of illustration, both an application running on a server and the server can be a component. One or more components can reside within a process and/or thread of execution and a component can be localized on one computer and/or distributed between two or more computers. In another example, respective components can execute from various computer-readable media having various data structures stored thereon. The components can communicate via local and/or remote processes such as in accordance with a signal having one or more data packets (e.g., data from one component interacting with another component in a local system, distributed system and/or across a network such as the Internet with other systems via the signal). As another example, a component can be an apparatus with specific functionality provided by mechanical parts operated by electric or electronic circuitry, which is operated by a software and/or firmware application executed by a processor. In such a case, the processor can be internal and/or external to the apparatus and can execute at least a part of the software and/or firmware application. As yet another example, a component can be an apparatus that provides specific functionality through electronic components without mechanical parts, where the electronic components can include a processor and/or other means to execute software and/or firmware that confers at least in part the functionality of the electronic components. In an aspect, a component can emulate an electronic component via a virtual machine, e.g., within a cloud-computing system.

The embodiments described herein include mere examples of systems and computer-implemented methods. It is, of course, not possible to describe every conceivable combination of components and/or computer-implemented methods for purposes of describing the one or more embodiments, but one of ordinary skill in the art can recognize that many further combinations and/or permutations of the one or more embodiments are possible. Furthermore, to the extent that the terms “includes,” “has,” “possesses,” and the like are used in the detailed description, claims, appendices and/or drawings such terms are intended to be inclusive in a manner similar to the term “comprising” as “comprising” is interpreted when used as a transitional word in a claim.

As used herein, the term “one of A, B, and C” shall be understood to mean “only A, only B, or only C,” and not a combination of A, B, and C.

As used herein, the term “one or more of A, B, and C” shall be understood to mean any one of A, B, or C, or any combination thereof, including multiple occurrences of each element. This includes, but is not limited to, the following configurations: only A, only B, only C, A and B, A and C, B and C, A, B, and C, as well as multiple instances of A, multiple instances of B, multiple instances of C, or any combination of multiple instances of A, B, and C.

As used herein, the term “at least one of A, B, and C” shall be understood to mean any one of A, B, or C, or any combination thereof, including multiple occurrences of each element. This includes, but is not limited to, the following configurations: only A, only B, only C, A and B, A and C, B and C, A, B, and C, as well as multiple instances of A, multiple instances of B, multiple instances of C, or any combination of multiple instances of A, B, and C.

As referred herein, “clinical trial” is a research study in which one or more trial subjects are prospectively assigned to one or more interventions (which may include placebo or other control) to evaluate the effects of those interventions on health-related biomedical or behavioral outcomes.

As used herein, “Artificial intelligence” or “AI” refers to the ability of a digital computer or computer-controlled robot to perform tasks commonly associated with intelligent beings.

The term “feature” as used herein, in relation to machine learning and pattern recognition, represents or refers to an individual measurable property or characteristic of a phenomenon. Features are usually numeric, but structural features such as strings and graphs are used in syntactic pattern recognition. The concept of “feature” is related to that of explanatory variables used in statistical techniques such as linear regression.

As used herein, the term “medical data” refers to data that include all that is included in clinical data, health data/information, and further, administrative data comprising insurance information, billing and insurance claims data, appointment and scheduling information, patient surveys, patient-reported outcomes, and patient satisfaction.

As used here, the term “feature vector/s” are representations of data used to capture the essential characteristics or attributes of input data in a format that algorithms can process. Each element in a feature vector represents a specific feature (or attribute) of the data, quantified in a way that is meaningful for analysis.

As used herein, “feature engineering” with reference to artificial intelligence refers to the process of using domain knowledge to create input variables, or features, that make machine learning algorithms work more effectively. This process involves selecting, modifying, or creating new features from raw data to improve the performance and accuracy of predictive models. Effective feature engineering may significantly enhance the ability of machine learning models to identify patterns and make accurate predictions by providing the models with the most relevant and informative variables.

The term “opioid use disorder (OUD),” refers to a medical condition characterized by compulsive drug-seeking behavior despite negative consequences. Severe cases may lead to opioid addiction, overdose, or death. Alternative terms include opioid drug abuse, opioid misuse, opioid dependence, opioid addiction, narcotic abuse, substance abuse, and prescription drug abuse.

The term “prescriber” is a licensed healthcare professional, such as a physician, nurse practitioner, or pain specialist, authorized to prescribe medications, including opioids.

The term “prescriber profiling” refers to analyzing prescriber related information, for example, assessing prescription patterns, drug preferences, frequency of opioid prescriptions, refill tendencies, and compliance with standard prescribing guidelines. This helps identify potential overprescription, deviations from best practices, or prescribers contributing to high-risk opioid use trends.

The term “subscriber” refers to an individual who receives a prescription for a controlled substance, such as opioids. This includes patients undergoing treatment for pain management or other conditions requiring opioid medications.

The term “subscriber profiling” refers to analyzing subscriber related information, for example prescription frequency, dosage levels, medication adherence, and multi-provider visits to detect patterns of overuse, misuse, or high-risk behavior.

The term “Decision Support System (DSS)” refers to a computer-based tool designed to assist users in making informed, data-driven decisions by analyzing large datasets, generating insights, and providing recommendations. It integrates various aspects such as data collection, data processing, predictive modeling, and rule-based logic to support decision-making across various domains, for example, healthcare.

The sources supporting certain data and statements presented in this business problem, business solution, technical problem section are listed in the references section.

### Business Problem:

The cost of the opioid epidemic in the US is staggering. From 2001 to 2016, direct costs associated with OUD and opiate abuse, including healthcare, social services, and lost wages/revenue topped $1 Trillion ($66B per year) with an additional $500B lost in the next five years ($100B/year). The problem is accelerating yet lacks proactive and predictive solutions. The entire care chain including manufacturers, distributors, prescribers, pharmacists, and patients all play a key role in this problem.

### Business Solution:

Due to the high rate of initial exposure via prescription medication, a product to aid physicians in the prediction and identification of those at risk of developing OUD is required. The business solution involves providing a suite of AI-driven tools that can be used to detect and/or prevent developing opioid use disorders.

### Technical Problem:

A retrospective analysis of over 375,000 Medicare beneficiaries treated by >14,000 physicians, found that opioid prescribing rates can vary greatly among clinicians (7.3% to 24.1%) for patients with overlapping clinical classification. High-intensity prescribing practices caused patients to experience significantly higher levels of chronic opioid use and developing OUD than patients treated by low-intensity prescribers. Arming prescribers as well as behavioral interventionists and pharmacists with data-driven insights can have a positive impact on the opioid epidemic. Physicians need a way to predict the likelihood of a patient developing OUD if prescribed an opioid and use that information to select a treatment paradigm that balances patient needs and the risk of deleterious downstream consequences.

Pharmacists can also act as a second line of defense in stopping OUD before a patient develops it. They can coordinate with prescribers to reconsider the therapeutic approach if armed with data-driven probabilistic insights, a recurrent theme in the literature. Within the last year Walgreens® and CVS® entered into a multi-state settlement deal totaling $10.7B and are thus highly incentivized to be proactive in the identification of those suffering from OUD or those who are at risk of doing so. In the most extreme circumstance, the pharmacist has the authority to refuse to fill prescriptions if they feel it will harm the patient. However, without data-driven quantifiable probabilistic reasoning, they are vulnerable to malpractice suits or accusations of biased care. As a leading review pointed out, “Due to specialized training in medication safety, management, and monitoring, pharmacists are uniquely qualified to participate in initiatives addressing appropriate management of prescription opioids; therefore, many governmental, healthcare associated, and pharmacy-associated entities require or encourage pharmacist participation in addressing the opioid crisis.” Even a single, short-term opioid prescription could lead to OUD. Therefore, it is imperative to give physicians and pharmacists the tools they need to develop and deliver the safest treatment plans to their patients by identifying those patients who have a high probability of becoming dependent on opioids.

OUD has disproportionately affected disadvantaged groups. Rates and treatment access differ by race, ethnicity, and income. The average annual percentage change (AAPC) of OUD rates for those who self-identify as black was double that of those who identify as white (26.16% increase versus 13.19% increase). Indigenous people and multiracial individuals suffered from OUD with the highest prevalence. Conversely, the uninsured and Asia Pacific (APAC) Americans most often underutilize opioid-specific treatment, with just 1.24% of these groups using opioid-specific treatment. To successfully control substance misuse, OUD, and diversion, a comprehensive solution must incorporate multiple risk factors that impact a patient's safety. Intervention must be data-driven to minimize bias and must occur at multiple levels of patient care including on a per patient basis and presented to both physicians and pharmacists. By identifying those patients who have a high chance of developing OUD, care providers will have definitive evidence to suggest a course of treatment that balances quality of life with risk of future negative consequences on multiple time horizons.

Prior art solutions have predictive analytics models that have been developed using artificial intelligence and deep learning algorithms. These algorithms behave like black boxes and cannot identify underlying risk factors but identify patients who are at risk of developing OUD. The AI-based deep learning or neural network models may be accurate, but they fail in identifying underlying factors and fail in explaining why a patient is at high risk. These models and algorithms are designed to constantly update outputs through the processing of new information but cannot identify the underlying causes of the OUD.

Therefore, there is a need for an innovative solution for predictive and prescriptive analytics to identify patients and prevent them from developing opioid use disorder. The present disclosure provides a mechanism to a formally described model, which tackles the above-mentioned challenges and provides systems and methods of predictive and prescriptive analytics to identify patients and prevent the development of opioid use disorder.

### Technical Solution:

To address the above OUD problems, the technological solution to identify patients and prevent developing opioid use disorder (OUD) is provided by integrating advanced technologies, such as explainable artificial and machine learning (AI/ML) models by predictive and prescriptive analytics for detection of the OUD in patients. Through the explainable model the program can identify highly contributing risk factors, which is then used for early intervention or treatment recommendations to deter addiction. The solution harnesses predictive analytics using artificial intelligence algorithms to generate an indicator, a probability score, of a patient developing OUD. Once potential addiction and an addict are identified along with contributing factors for such addiction, prescriptive analytics, which is built using machine learning models, provides treatment recommendations to assist health professionals in making more informed decisions related to drug dosage and overall approach to treatment. This solution also provides health professionals with an opportunity to intervene, such as treat at-risk patients with craving-suppression medication. The benefits to individuals and to society can be significant, not to mention the cost savings related to early prevention treatment and rehabilitation. Physicians may prescribe the treatment as recommended by the system.

According to an embodiment, it is a unique two-phased solution that provides an individualized patient prediction and prescription for preventing Opioid Use Disorder (OUD). This two-phased solution uses the following methodologies:
(a) Predictive analytics for identifying patients who are at the risk of developing OUD along with underlying attributes contributing to the risk, and
(b) Prescriptive analytics for providing treatment recommendations; and
(c) Improving clinical trials on OUD patients using predictive analytics.

In one embodiment, modules (a), (b), and (c) may function independently, with the output of one module serving as the input for the next. In an embodiment, any of the modules could be deployed separately without the need of the other. For instance, the output of module (a) could be fed into module (b), and the output of module (b) may then be used as the input for module (c). In another embodiment, these modules could be integrated into a single system, collectively processing data to generate a final output based on the analysis performed across all three modules.

### Technical Details Specific to the Technical Solution:

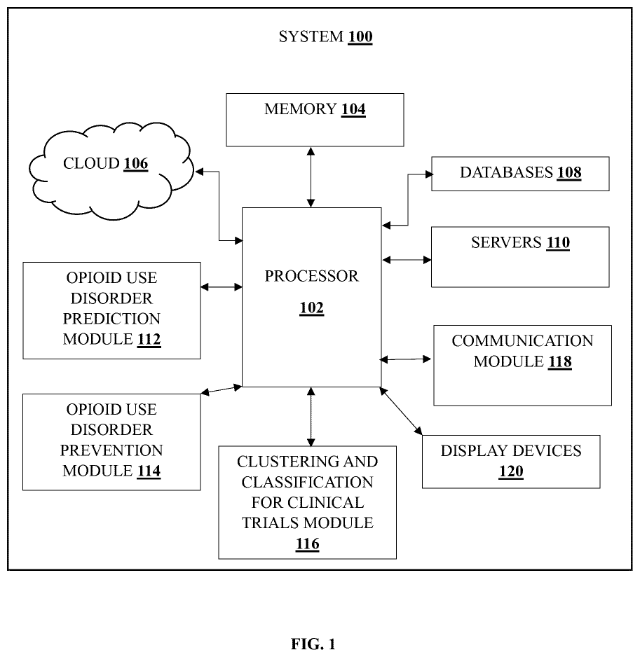

FIG. 1 illustrates a system for identification and prevention of OUD in patients using OUD Risk Tool, according to one or more embodiments.

FIG. 1 shows system 100 which may comprise processor 102, memory 104 which may be linked to cloud 106, one or more databases 108, one or more servers 110, opioid use disorder prediction module 112, opioid use disorder prevention module 114, clustering and classification for clinical trials module 116, communication module 118, and one or more display devices 120. The processor 102 may further be linked to or interacting with databases 108, servers 110, clustering and classification for clinical trial module 116, communication module 118, and display device 120.

Processor 102 may be a high-performance, multi-core CPU or system-on-chip (SoC) solution to process vast amounts of data. In some embodiments, processor 102 processes data from memory 104, database 108, and other modules connected to the processor. Processor 102 may comprise Graphics Processing Units (GPUs). GPUs are utilized for their ability to accelerate tasks like data processing. The processor may depend on aspects such as processing requirements and power consumption. Processors, also known as central processing units (CPUs), are the heart and brain of any computer or electronic device capable of executing instructions. Processor or processors' function is to process data and perform calculations, etc. At the core of their operation lies data processing, where they handle arithmetic and logical operations on data stored in memory. CPUs execute instructions, which are sets of specific operations encoded in machine language, to perform various tasks. The control unit within, or interacting with, the processor manages and coordinates the execution of instructions, fetching them from memory, decoding them, and directing the appropriate components to execute the instructions. To ensure a controlled and orderly flow of tasks, processors use an internal clock that generates regular electrical pulses, synchronizing their operations through clock cycles. Processors support multitasking environments, rapidly switching between executing different tasks for various applications. Additionally, they may work with the operating system to manage virtual memory, allowing programs to access more memory than is physically available, and to efficiently manage memory usage. Processor or processors may be integrated with security features, including hardware-level encryption, memory protection, and support for secure execution environments, enhancing the system's security against potential threats. The processor may run sophisticated algorithms and artificial intelligence (AI) software to analyze input data, interpret the environment, and help in decision-making. In an embodiment, the processor may be a neuromorphic processor, inspired by the human brain, which offers a unique approach to handling AI tasks. Processor 102 interacts, exchanges data, controls, and coordinates with one or more of the other components or modules of the system, computes, processes, and provides an individualized patient prediction and prescription for preventing Opioid Use Disorder.

Memory 104 may be a non-volatile memory (NVM) which is utilized in reliable operations of the system, ensuring that data is preserved even during power interruptions or failures. Various NVM technologies are utilized, such as flash memory for storing the operating system and software, EEPROM for retaining configuration data, calibration values, and sensor settings, Ferroelectric RAM (FRAM) for critical real-time information, and emerging technologies like ReRAM for potential performance enhancements due to their high-speed operation and low power consumption. In an embodiment, the memory may be a cloud-based memory. In another embodiment, the memory may be a local memory. In another embodiment, the memory may be a combination of local and cloud-based memory. Local memory refers to the traditional memory components present in a physical device, such as a computer's RAM, hard disk drives (HDDs), or solid-state drives (SSDs). It provides fast access to data, making it suitable for immediate processing tasks and offline use. On the other hand, cloud-based memory relies on remote servers and services provided by third-party cloud providers to store and manage data over the internet. Systems can access their data from anywhere with an internet connection, allowing for seamless collaboration and scalability. Cloud-based memory is often used for storing large amounts of data, enabling data sharing, and providing backup and disaster recovery solutions. The combination of local memory and cloud-based memory allows for flexible and efficient data management tailored to different needs of the system.

The system may access data from one or more databases 108. Databases can include electronic health records (EHRs) and prescription drug monitoring programs (PDMPs).

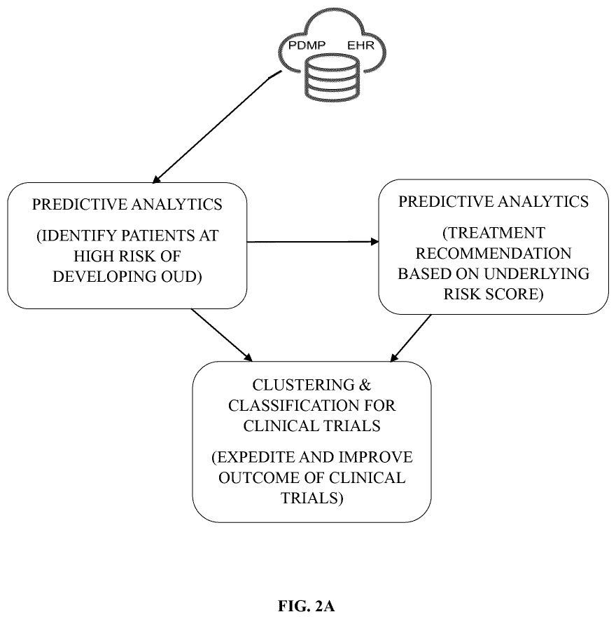

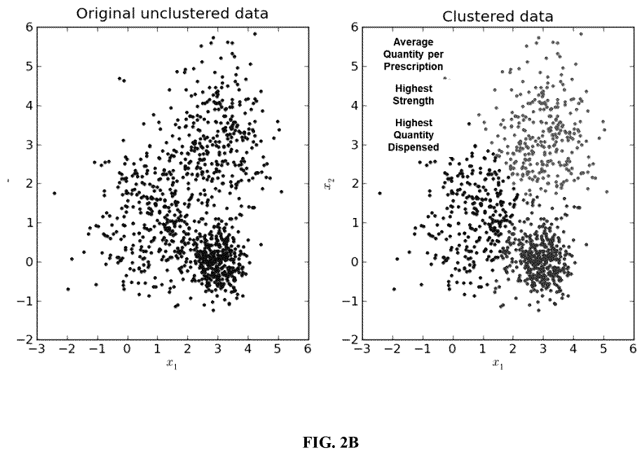

Servers 110 may be a computer or system that provides resources, data, services, or applications to other computers, known as clients, over a network. Servers are used in computing by handling requests and delivering responses in various applications, such as OUD applications for various clients. One of their functions is data storage and management, where they store, process, and organize data for users and applications, as seen in database servers. Additionally, servers are used for hosting websites and applications, ensuring that web pages and online services are accessible over the internet through web servers. Another use involves handling client requests, where servers receive, process, and respond to requests, facilitating smooth communication, as in Application Programming Interface (API) servers. Furthermore, servers contribute to security and authentication by managing user authentication, access control, and encryption to ensure secure data access, as seen in authentication servers. They also support resource sharing, allowing multiple users or systems to share computational power, storage, or network access, as in file servers and cloud servers. Depending on their purpose, servers can be dedicated, where they handle specific tasks, or shared, where they manage multiple services, and they may operate on-premises or in cloud environments. In one example, the servers have a speed in the range of 2 to 3 GHZ, have 4 to 16 cores, and a memory capacity of 12 to 15 GB. However, the parameters may be different, depending on the capacity requirements. The system may also include technologies for collection, processing, storage, and distribution of data such as Smart Phones, iPads, Desktop/Personal Computers, Stand-alone/On-Premise/Cloud Servers, and the like. Each device and server comprise digital data processors and communication interfaces as is well-known in the art. Opioid use disorder prediction module 112, opioid use disorder prevention module 114, clustering and classification for clinical trials module 116 may interface with databases 108 and apply one or more algorithms to provide trends and predictions regarding OUD and prevention strategies. These modules, opioid use disorder prediction module 112, opioid use disorder prevention module 114, clustering and classification for clinical trials module 116, are further elaborated in FIG. 2A. The outcome, such as the trends over a period of time and the predictions may be displayed on the display device 120. In some embodiments, database 108 may be locally present in system 100 or may be present in cloud 106. The data stored in the databases 108 may be fetched and displayed on the display device 120. The system may have various components with a suite of Artificial Intelligence algorithms developed using any suitable software. The database 108 may comprise a data anonymization unit which anonymizes or pseudonymizes the inputs received by discarding the metadata, such as patient details, associated with the inputs. Anonymization may be performed to break the link between data and a given participant so that the participant cannot be identified, directly or indirectly. Such an anonymization can be performed based on rules which are preconfigured or configured at the time of data transfer. The patient's details may contribute to determining the identity or recognizing the patient. One or more of patient identifiable fields, such as, but not limited to, patient name and patient ID, address, social security number, credit card information, etc., may be anonymized based on the purpose of the use. Once the data is anonymized, the patient identity is concealed such that one cannot trace or track the source (e.g., patient identity, site identity, etc.) of the medical data. In an embodiment, the data anonymization unit anonymizes the inputs by removing facial detection information and biometrics information from the inputs, if present. The database technology may be instantiated as a Relational Database Management System (RDBMS) or as an in-memory data grid spanning clusters of servers to allow for faster throughput and real-time processing of events as they occur during the predictive and prescriptive analytics methodology. The deployment of the database(s) may be in a private data center on a secured public cloud infrastructure to allow for quick scale up during periods of intense activity where the volumes of data approach that of a data stream and may require additional infrastructure to support spikes in demand during these periods. Designing and developing such a complex computation system for predictive and prescriptive analytics utilizes a software ecosystem/platform with robust computational infrastructure.

Communication module 118 comprises hardware that facilitates the transmission, reception, or exchange of data between systems, individuals, or networks. These devices enable connectivity across various platforms, whether through wired or wireless technologies. In an embodiment, a communication module may be used to send alerts to the users of the system. Communication module 118 may send alerts to patients, physicians prescribing the drug, and pharmacists as needed. Communication alerts for opioid use disorder are sent to ensure patient safety, preventing misuse, and enhancing coordination among healthcare providers. For a patient, alerts can include medication adherence reminders, which notify individuals to take prescribed opioid medications as directed, or risk alerts, which warn about potential overdose risks when multiple opioids or sedatives are prescribed. Additionally, patients may receive educational notifications about safe opioid use, disposal methods, or the availability of medicines for overdose reversal. For a prescriber, alerts can include prescription monitoring system (PMP) notifications, which flag cases where a patient is receiving opioid prescriptions from multiple providers, indicating possible misuse. Dosage limit warnings can notify prescribers when an opioid prescription exceeds recommended guidelines, reducing the risk of overdose. Drug interaction alerts can inform healthcare providers when an opioid prescription may cause harmful interactions with other medications that the patient may already be taking or in general. For a pharmacist, communication alerts can involve duplicate prescription warnings, which highlight instances where a patient has been prescribed opioids from different providers. Early refill alerts can notify pharmacists when a patient is attempting to refill an opioid prescription too soon, indicating potential misuse. Mandatory counseling alerts can prompt pharmacists to provide opioid safety education, such as explaining the risks of dependence and proper storage methods to prevent misuse or diversion. These communication alerts are integrated into the system to enhance patient safety, promote responsible prescribing, and reduce the risk of opioid misuse and overdose.

FIG. 2A illustrates various modules of the system for opioid use disorder prediction and prevention according to an embodiment. The system comprises predictive analytics to identify patients at high risk of developing OUD, prescriptive analytics to provide treatment recommendation based on a risk score from the predictive analytics module, and cluster and classification for clinical trials to expedite and improve clinical trials, according to an embodiment.

The predictive analytics methodology uses a probabilistic (logistic) model that generates a score that determines the probability or likelihood of a patient developing OUD. The prescriptive analytics methodology uses a machine learning algorithm that uses the underlying risk factors from the predictive analytics and provides treatment recommendations to health professionals on an individualized basis. The approach provided in the present disclosure uses a logistic model that is explainable and can pinpoint to specific attributes that contribute towards higher risk scores.

The input to the model is from the PDMP and EHR data sources, which is stage one of the architecture. The logistic model is used for the predictive analysis to identify patients at risk of developing OUD. There are multiple attributes considered from the PDMP and EHR data sources. The probability score is calculated or computed by the model and is further used by the prescriptive analytics model, and the prescriptive analytics recommends a treatment plan out of the available options.

By identifying patients who are at risk of developing OUD and then using the underlying attributes (or risk factors or causes) to determine treatment recommendations, i.e., combination of predictive and prescriptive analytics is a holistic approach that is designed to improve diagnostics, accuracy in treatment, and early intervention. This newly designed approach exceeds a physician's expertise and provides practitioners with the most suitable and advanced techniques to improve medical practices in the areas of diagnosis, treatment recommendations, and prognosis.

The disclosed solution harnesses predictive and prescriptive analytics using artificial intelligence algorithms, in particular probabilistic models, to generate an indicator (a probability score or a risk score). Once an individual is identified as having potential OUD, the prescriptive analytics models, which are built using machine learning, provide treatment recommendations to assist health professionals in making more informed decisions related to drug dosage, as well as alternative medication options. This solution also provides health professionals with an opportunity to treat at-risk patients with craving-suppression medication.

The disclosed solution leverages prescriptive analytics to not only identify individuals at risk for opioid use disorder (OUD) but also to provide actionable treatment guidance tailored to each patient's condition. Once predictive analytics identifies a patient with a high probability or risk score for OUD, prescriptive analytics models, trained using machine learning on vast datasets of clinical histories, treatment outcomes, and medication responses, and recommend precise treatment interventions. These recommendations may include personalized drug dosage adjustments, alternative non-opioid pain management strategies, and the introduction of medication-assisted treatment (MAT) options, based on the patient's medical history, genetic predisposition, and response to past treatments.

Additionally, the system of predictive analytics and prescriptive analytics can be integrated into clinical decision support systems (CDSS), where the AI-driven recommendations are directly embedded into electronic health record (EHR) systems or Prescription Drug Monitoring Program (PDMP) systems, allowing physicians, pharmacists, and other healthcare providers to access real-time insights during patient consultations. The system, upon flagging an OUD, may suggest dose tapering schedules, flag potential drug-drug interactions, and offer alternative pain management therapies, such as physical therapy, cognitive-behavioral therapy (CBT), or non-opioid analgesics, depending on the individual's risk profile.

Furthermore, this solution enhances early intervention capabilities by providing alerts for at-risk patients, enabling health professionals to proactively prescribe craving-suppression medications before addiction escalates. It also facilitates ongoing patient monitoring, allowing clinicians to track patient adherence to prescribed treatments and adjust care plans dynamically, ensuring optimal therapeutic outcomes while minimizing the risk of opioid misuse, dependence, or overdose.

Predictive analysis and Prescriptive analytics recommendations could be integrated into patient care systems through electronic health records (EHRs), clinical decision support systems (CDSS), and pharmacy management platforms to assist healthcare providers in optimizing treatment decisions. When a patient is flagged as high risk for opioid use disorder (OUD), the system generates actionable recommendations tailored to the patient's medical history, current medications, and risk profile. For physicians, these recommendations may appear as real-time alerts within the EHR, suggesting dose modifications, alternative pain management therapies, or medication-assisted treatment (MAT) options. For instance, if a patient with chronic pain is identified as high risk for opioid dependence, the system may advise switching from an opioid prescription to non-opioid analgesics, physical therapy, or behavioral interventions.

For pharmacists, the recommendations integrate into pharmacy dispensing systems, where alerts may notify them of potential drug-drug interactions, high-dose opioid prescriptions, or frequent refills. If a patient attempts to fill multiple opioid prescriptions from different providers, the system may trigger a warning, prompting the pharmacist to review the prescription history and consult with the prescribing physician.

Further, the system may generate alerts for identified patients with OUD. Notifications can be sent via EHR pop-ups, email alerts, SMS (Short Message Service) notifications, or automated system flags to ensure timely intervention. These alerts help prevent opioid misuse by prompting healthcare professionals to take appropriate actions, such as initiating a patient consultation, adjusting treatment plans, or referring the patient to addiction specialists.

In an embodiment, there are methods designed and implemented for data transfer mechanisms and interdependencies among predictive, prescriptive, and clustering/classification models. In an embodiment, methods for data translation, integration, or transformation across models are designed and implemented to generate final outputs.

In an embodiment, the predictive model and the prescriptive model could be separate models. The predictive algorithm first identifies the patients who are at higher risk of developing opioid use disorder. The output is fed to the second model, which is the prescriptive analytics, that provides treatment recommendations based on the risk score and underlying explanatory variables or underlying conditions.

FIG. 2B is an illustration of unsupervised machine learning algorithms to grouping patient data into clusters according to an embodiment. Prescriptive analytics uses unsupervised machine learning algorithms to group patient data into clusters based on similarity in the risk factors. All the patients with probability higher than 50% of developing OUD are clustered using the K-Mean Clustering algorithm.

For each of the underlying risk factors with higher weights, such as—a) Average Quantity per Prescription, b) Highest strength, c) Highest quantity dispensed, d) Average duration between refills, e) Total prescription in last six months, K-Mean Clustering algorithm is used to group all the patients. The risk factor that contributed highest (or top two risk factors if they are close in value) is used to identify a particular cluster by identifying the closest centroid.

Machine learning algorithms comprising Large Language Models learn and develop a set of recommendations for each of the clusters. Prescriptive analytics, also referred to as recommendation engine, will use this knowledge to guide prescribers and subscribers with treatment recommendations based on the cluster a particular patient belongs to.

For example, if there were 50,000 patients in a cluster in the past, and when the changes were made to the treatments of the patients based on the treatment recommendations provided by the model, and the patients had a positive impact, the risk scores went down. In this case the prescriptive analysis analyzed the data collected on all the other patients and analyzed how the changes worked for those patients.

In another example, consider there are 5 million records, or 5 million patients that are monitored, and suppose they are clustered into 10 different categories. When a 5,000,001 patient came, the system can look at the clusters and identify a cluster to which this patient belongs, based on the risk score and a subset of the underlying conditions, and then the system can analyze the cluster and provide the analysis, for example, when a set of changes were applied to this patient in prescription or an alternate medicine was prescribed, there was a positive response, meaning the probability or risk score went down. The system is informing the doctor that on this 5,000,001^{st} patient, providing a prescription, or treatment recommendations as analyzed and output by the system, will work, because the system knows enough about patients belonging to that cluster. The cluster could be based on many factors such as the data attributes from the PDMP and EHR records. The clustering algorithm may use recursive clustering or recursive partitioning, such as tree clustering, or random forest clustering.

An application of the model further is used to improve clinical trials on OUD patients using predictive analytics. For clinical trials, the input to the model may be the same as for predictive analytics, the PDMP and EHR data sources. For clinical trials, the application of the model is to accelerate patient enrollment, as for a clinical trial the predictive analytics mentioned in the disclosure may be extended for use in identifying target groups and patients for enrollment in clinical trials. This helps target a particular group, having the required threshold probability, to enroll in clinical trials.

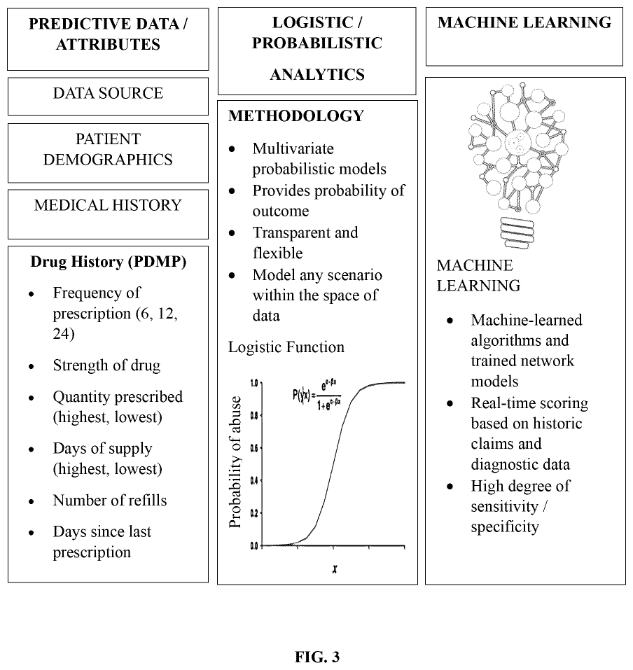

FIG. 3 illustrates a predictive analytics module according to an embodiment. The goal in using predictive analytics is to prevent patients from developing OUD by identifying and treating patients based on each of their unique circumstances and medical history through early intervention techniques. The benefits to individuals and to society can be significant by not only helping to prevent the need for rehabilitation, especially considering the high recidivism rates (According to the National Institute on Drug Abuse, 85% of individuals relapse within one year of treatment), but also for the cost savings related to hospitalizations, rehabilitation, and society as a whole.

Predictive Analytics: A system is developed, for predictive analytics using statistical regression techniques, to provide a unique solution that uses probabilistic models to generate an indicator (or probability score) that can identify a patient carrying a high-risk score. Predictive analysis comprises a multivariate logistic regression, which comprises an analysis of heuristically selected features (explanatory/independent variables) to calculate the probability of a given patient being an addict.

To identify patients who have a high risk of developing OUD, logistic models are used to characterize a response variable that represents a probability-like score of the target activity. The solution is implemented by applying the logistic distribution to a linear, weighted combination of features to produce an estimated probability between 0 and 1. The weights of the equations are trained and recalibrated using statistical regression over a selected training set/training dataset.

The process to develop and implement a probability score has two phases:
(a) Data derivation and summarization is used to derive each explanatory variable or attribute data from the raw data. Based on the characteristics of the raw data, using rule-based algorithms, various statistical techniques are applied to normalize the data by reducing the long tails or converting unstructured data, as described below, into categorical variables.
(b) Logistic models are used to calculate the probability score of a patient developing OUD.

The logistic/probabilistic model (analysis) in the disclosure comprises a multivariate logistic regression, which comprises an analysis of heuristically selected features (explanatory/independent variables) to calculate the probability of a given patient being an addict.

A Θ (Theta) equation is contrived to produce a value used for calculating the logistic score. This equation is of the form Θ_{i,j,k}=C₀+Σ₁ⁿCi*Fᵢ. Each Cᵢ is the regression coefficient or relative weight of each feature, while each Fᵢ is the value of each feature. The logistic score is then calculated using the logistic function

$$L = \frac{1}{1 + {e}^{- Θ}}$$

where Θ is the output in the previous equation. These equations produce a pseudo-probability, which behaves similarly to non-normal independent variables. This outcome score may refer to the probability of the patient being readmitted within 30 days of discharge. This probability score is then used to conduct a clinical review and prepare a test dataset to train the AI model to produce more accurate results for each patient.

The goal from these analytics is to provide a recommendation to practitioners for each patient that is treated for pain and is prescribed an opioid prescription. The analytics, over a period of time, can be enhanced to profile practitioners and their behavior as well.

The machine learning algorithms use various clustering methodologies to provide valuable insights into each patient and are designed to generate treatment recommendations. Along with these recommendations, the OUD probability score can also be used by physicians as a tool to assist in making more informed decisions related to dosage, alternative medications, as well as craving-suppression medications.

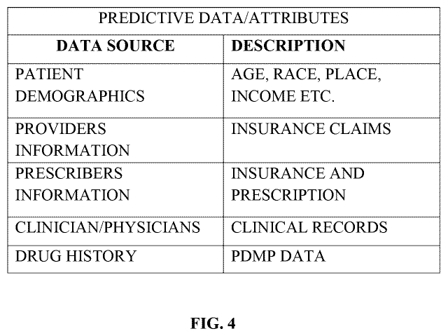

FIG. 4 illustrates predictive data/attributes and the source from which the data is collected according to an embodiment. FIG. 4 shows the data source and the description of the predictive data or the attribute. Predictive data comprises patient data, prescription data, drug data, dispenser data and prescriber data. This data is collected from the sources such as patient demographics data from electronic health records, providers information from insurance claims, prescribers' information from insurance and/or prescription, clinician and physician information from clinical records, drug history from PDMP databases.

Usually, the input data is obtained from multiple sources, multiple databases and the data formats vary between sources depending upon how the data is stored and organized. The input data may be inaccurate, noisy, and inconsistent due to the nature of data acquisition processes and diversity of the nature of data. Therefore, the system runs one or more preprocessing algorithms that deal with processing both structured and unstructured data. These algorithms preprocess the structured and unstructured data and then forward the cleaned data to the subsequent modules for further processing and analysis. Some of the preprocessing approaches are:

### In Structured Data:

Data Inaccuracy—handling the incomplete, missing values can be done using traditional techniques such as imputation with mean, normal values and also with model-based approaches such as multivariate regression and k-nearest neighbor. Data Noise—reducing noise by removing erroneous data and outliers from the data by multivariate approaches.
Data Inconsistency—identified when data is input from various sources. During this time, the source with the most inconsistent data can be identified and can be addressed using correlation analysis.

### In Unstructured Data

Notes/Text: For textual data, the normalization can be a task for analysis of clinical staff's notes and laboratory reports. With normalization, the system handles some of the challenges in text processing such as:
Format/Code Conversion—data from multiple sources in various formats/codes can be collected and converted to simple format. The system incorporates Scripts for converting files in different formats to one standard format.
Eliminating Stop Words/Punctuations/Non-ASCII characters—The system incorporates regular expression scripts to eliminate the stop words, punctuations and non-ascii characters.
Identifying Stem Words—reducing each word in the text to base or root improves the analysis of textual data. The system comprises modules for performing stemming on clinical and laboratory notes.
Lemmatization—as used herein can refer to reducing words to base form by considering the context, along with the content, and can be useful in identifying clinical, biological entities in notes or reports. Alternatively, lemmatization of words helps to tag the text.

EDA—Exploratory Data Analysis or Exploratory Models: In such models, the system also considers the synthesized results pertaining to the factors associated with individualized patient prediction and prescription for preventing Opioid Use Disorder. Such an exploratory analysis can be used in prescriptive analytics for providing treatment recommendations. Some of the visualizations rendered in the dashboard could be depicted using the charts, speedometer, gauge meter, and/or horizontal bar charts.

Representation Learning: Performance of the prediction model depends upon the quality of data pooled for training the model. Deep neural network models are trained to learn data representation for the data considered as the input. To improve the performance of the prediction model, vector representation can be adopted to denote the content in the data. Furthermore, information extracted from the various sources of data is also combined with the other characteristics of the data and are represented as vectors.

Learning & Extracting from Text Data: The AI suite of the system has neural network models of type recurrent neural networks to perform the task of extracting information from the unstructured data such as lab reports, staff's notes, etc. Models can be trained to identify the clinical concepts in text and map them to the standard clinical approaches. Thereby trained models enable transformation of unstructured text into information represented in vectors.

Learning & Extracting from Image Data: The AI suite of the system comprises deep neural network models of type convolutional neural networks to perform the extraction of information from different types of scans such as ultrasound, Magnetic Resonance Imaging (MRI), etc. Networks are trained to learn object segmentation from the scanned images. Once trained, the model has the ability to detect objects from the knowledge it has gained about image features. Upon extraction of the object from the scanned image, information about the properties of the object is represented in vectors.

Information extracted from deep neural networks can then be passed to the stacked neural networks with deep hidden layers. These layers have a large number of nodes with non-linear activation functions and thus have the ability to capture the non-linear association with the various data characteristics of the predictive analytics. Projection of the risk element characteristics to the higher dimension enhances the opportunity to better understand the association between different characteristics. Training of the model may be done in the context of supervised learning.

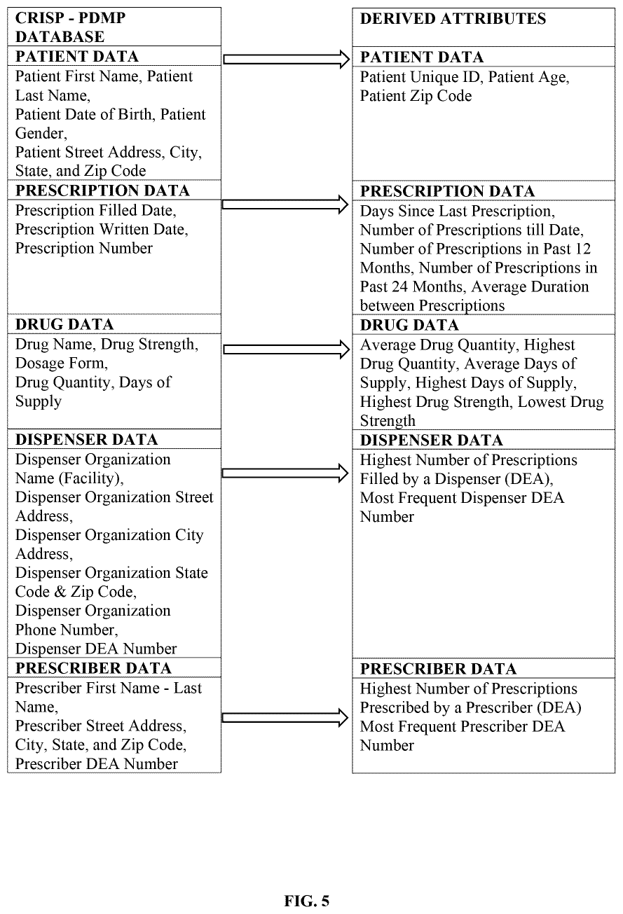

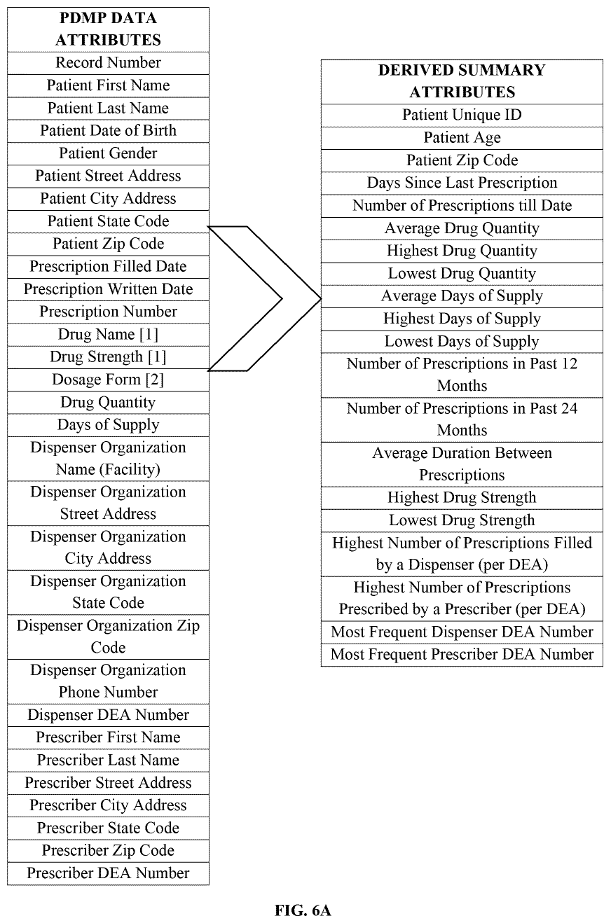

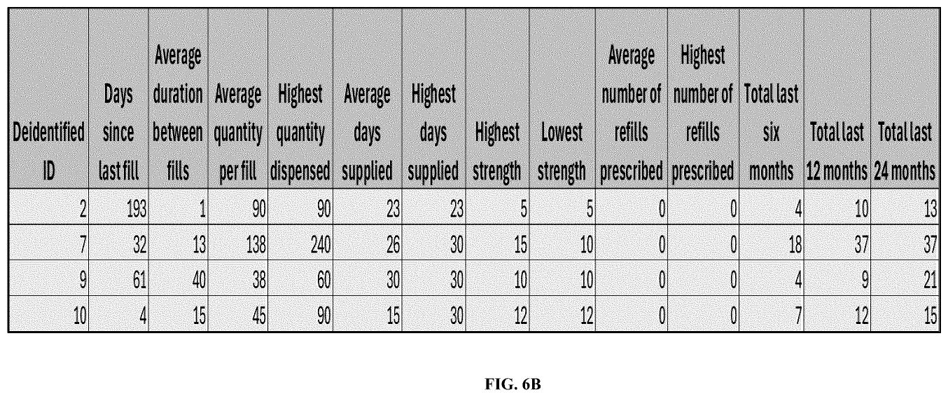

FIG. 5 shows raw data taken from the PDMP database and derived attributes according to an embodiment. FIG. 6A illustrates Prescription Drug Monitoring Program (PDMP) Data attributes used in predictive analytics and prescriptive analytics according to an embodiment. FIG. 5 and FIG. 6A shows raw data and explanatory variables (or attribute data) derived from the raw data according to the raw database. Based on the characteristics of the raw data, using rule-based algorithms, various statistical techniques are applied to normalize the data by reducing the long tails or converting unstructured data into categorical variables.

Prescription Drug Monitoring Program (PDMP) Data Attributes used in analytics, left-hand side data in FIG. 5 and left-hand side data in FIG. 6A is the raw data that could be a super set data, and on the right-hand side of each of these figures is the derived data per patient. The attribute data is also referred to as explanatory data or input data and is derived and summarized for each patient in the database from the raw data of the patient. In an example, one patient can have, over the last five years, say for example 60 records, one for each month the patient got an opioid prescription. That data is considered the raw data. The data is then summarized per patient, since the model is built per patient, not per prescription. The raw data is summarized at the patient level, and that becomes the updated data source. A few examples of the attributes or features may include the number of prescriptions the patient had in the last twelve months, average number of prescriptions per period, the maximum quantity of dosage per prescription, the details of when the last prescription was filled, such as the number of days passed since the last prescription, the average duration of prescription, the highest drug strength, etc. These attributes have the potential to be a part of the predictive model.

In an embodiment, PDMP and clinical data from 1866 patients were used to prospectively predict the likelihood of OUD development, current OUD status, and retention in care of PWH. Overall, it is found that the models correctly predicted OUD development and those currently suffering from OUD with 89% correct classification, a level that is groundbreaking for the field.

FIG. 6B illustrates a table showing all the prescription data over the last 24 months summarized or aggregated to calculate attribute values for each patient according to an embodiment. Once the raw data from PDMP and EHR are collected, they are summarized or aggregated for each patient. For example, as shown in FIG. 6B for each patient all the prescription data over the last 24 months are summarized or aggregated to calculate
a. Number of days since the last prescription was filled.
b. Average duration between the two prescriptions or refills.
c. Average quantity per prescription.
d. Highest quantity prescribed or dispensed.
e. Average days of supply, highest and lowest strength prescribed to the patient during 24 months.
f. Number of prescriptions within the last six, twelve and twenty-four months.

Every time a new prescription is entered in the PDMP and/or EHR, the summarization process recalculates all these attributes for that specific patient. Once the data are summarized, each attribute (for that patient) is analyzed and translated into categorical values to make sure their distribution is normal.

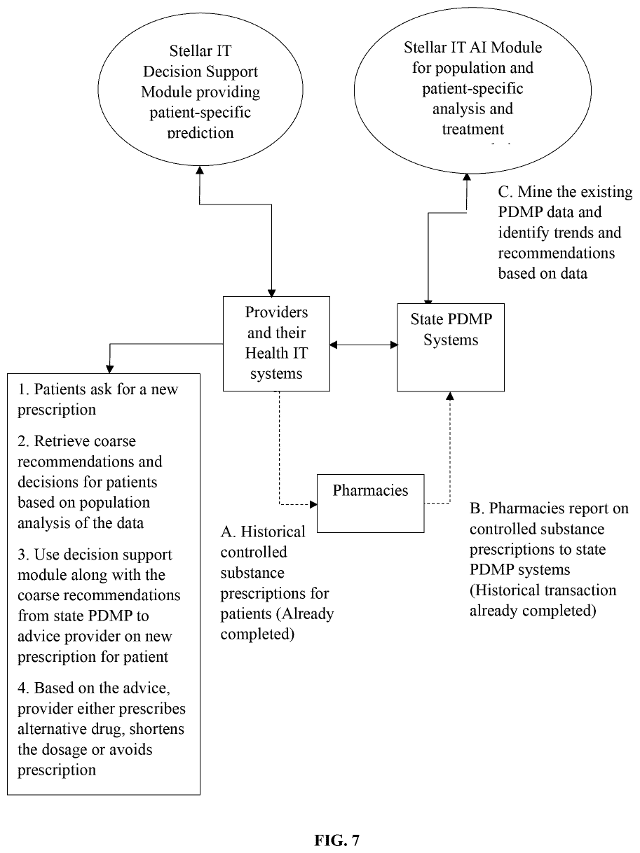

FIG. 7 illustrates Decision Support System for providing patient-specific prediction and treatment prescription, according to an embodiment.

For example, a patient is visiting their physician and asking for an opioid prescription for pain. The physicians or the providers use their system to pull the patient's PDMP record, which then connects them to the state PDMP system, and the physician will have access to the patient's risk score for the development of OUD. Thereafter, the physician can make the decision on the prescription, for example, in terms of the strength of the dosage, the number of pills, whether it is a seven days or 30 days or 60 days course, etc. This decision is made based on the risk score. Once the prescription is written, it is updated in the PDMP system. When the patient is taking the prescription, and then goes to the pharmacist for a refill, the pharmacist also pulls up the PDMP record to validate the prescription, and then they fill the prescription and update the details in the PDMP system, which is the master source of all the information. In an embodiment, the predictive and prescriptive analytics modules are integrated with the PDMP system. In an embodiment, the predictive and prescriptive analytics modules are integrated with an EHR system.

In an embodiment, the disclosed model updates the risk score of a patient every single night, at a prescribed time, or whenever there is a change in the underlying data, such as a change in the health condition or a new prescription is added in the PDMP for the patient. Therefore, whenever a change happens it gets updated and the model runs every night, takes into consideration the changes, if any, made for each patient and calculates the risk score. Anytime a provider or a physician writes the prescription, they access the PDMP data, and every time a pharmacist fills the prescription, they add the PDMP data, which has an updated score for each patient. Therefore, the risk score for a patient for the very first time they go for opioid prescription is zero and over a period of time the risk score may change, and the system predicts whether the patient is at risk of getting addicted or not.

### Decision Support Module Providing Patient-Specific Prediction:

### Substance Overdose and Abuse Prevention System

Opioid Use Disorder Prediction is a predictive analytical solution that can identify a patient developing OUD with a certain probability or likelihood. This solution harnesses predictive and prescriptive analytics using artificial intelligence algorithms to generate an indicator (a probability score). Once an addict or potential addict is identified, prescriptive analytics, which is built using machine learning models, provides treatment recommendations to assist health professionals in making more informed decisions related to drug dosage and overall approach to treatment. This solution also provides health professionals an opportunity to treat at-risk patients with craving-suppression medication, behavioral interventions, therapy, withdrawal reduction treatment, and educational materials to help patients overcome OUD.

Predictive analytical solutions in the healthcare industry are focused on predicting probability or likelihood of developing OUD of commonly prescribed opioid medications. The system of the current disclosure can identify patients who carry a higher risk of addiction through probabilistic models (statistical models) that can explain the outcome and identify underlying factors (attribute variables) that contribute toward high probability.

### Population and Patient-Specific Analysis and Treatment Recommendation:

### Overview of Substance Abuse Prevention Model

Prescriptive Analytics: Prescriptive analytics provides treatment recommendations to health professionals for each patient that is being identified with a probability score through predictive analytics. The analytics, over a period of time, will be enhanced to also profile health professionals and their behaviors.

Prescriptive analytics uses various clustering algorithms, such as decision trees or random partitioning, and enables the identification of distinct patient subgroups based on shared characteristics, risk factors, and treatment responses. Decision trees create hierarchical structures that segment patients based on key variables, such as demographic data, medical history, and prescription patterns, allowing for rule-based classification and treatment stratification. Random partitioning methods, including k-means or random forests, group patients into clusters without predefined categories, enabling the discovery of hidden patterns in patient data. By applying these clustering techniques, healthcare systems can generate personalized treatment recommendations tailored to the specific needs of each patient subgroup, optimizing drug dosages, alternative therapies, and behavioral interventions. Additionally, population-level clustering provides insights into regional or demographic treatment trends, helping policymakers and healthcare providers design targeted interventions to improve overall patient outcomes and mitigate risks associated with opioid use or chronic disease management.

The machine learning algorithms use various clustering algorithms to provide valuable insights into each patient and are designed to generate treatment recommendations. Along with the recommendations, the addiction probability score can also be used by physicians as a tool to assist in making more informed decisions related to dosage, alternative medications, as well as craving-suppression medications.

Predictive and AI Model Implementation with Clinicians and Patients:

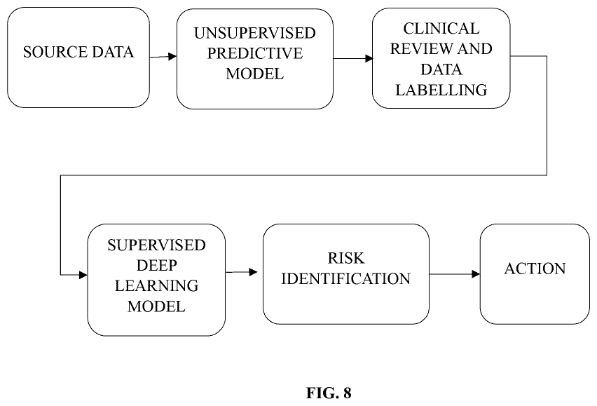

FIG. 8 shows the process steps involved in development and implementation of the proposed solution according to an embodiment.

### AI Model Development and Implementation Steps

Source Data: This is the first stage in this process, which summarizes and derives a patient's data that can be used in unsupervised predictive modelling. This data derivation is completed from the raw patient data. In this stage, various methods are used to assure that the derived attributes fit in the logistic model to provide better prediction.

Unsupervised Data Analytics: Once the explanatory variables are derived from the data, the program can run through the probabilistic model. In an embodiment, the predictive model could be an unsupervised model. In an embodiment, the predictive model could be a supervised model.

Clinical Review and Data labeling: After generating the probability score, the clinical experts review the cases and validate the classifications to prepare a trained dataset to feed into the supervised learning.

Data Summarization and Derivation: To identify patients with a high risk of addiction, logit models are used to characterize a response variable that represents a probability-like score of the target activity. The solution is implemented by applying the logit distribution to a linear, weighted combination of features to produce an estimated probability between 0 and 1. The weights of the equations are trained and recalibrated using statistical regression over a selected training set.

The process to develop and implement a probability score has two phases as explained herein elsewhere:
Data derivation and summarization is used to derive each explanatory variable from the raw data.
Logistic models are used to calculate the probability score of a patient being an addict or becoming an addict.

Supervised Deep Learning Model: In an embodiment, a supervised deep learning model is derived after clinical review and data labeling. The predictive model could be recalibrated based on the clinical review data. In the AI/ML model, weights are the adjustable parameters that determine how input data influences the output. During training, the model learns optimal weight values to minimize prediction errors. In neural networks, weights are associated with connections between neurons and control the strength of signals passing through the network. Each input feature is multiplied by a corresponding weight, and the weighted sum is passed through an activation function, such as the sigmoid, to produce the output. Initially, weights are set randomly or using initialization techniques like Xavier initialization or He initialization to improve convergence. During training, an optimization algorithm-typically gradient descent or its variants-adjusts the weights based on the loss function, which measures how far the model's predictions are from actual values. The adjustments are guided by backpropagation, a process that calculates the gradient (derivative) of the loss function with respect to each weight and updates them to reduce the error.

Risk identification (or Probabilistic Scoring): Statistical analysis comprises a multivariate regression, which comprises an analysis of heuristically selected features (explanatory/independent variables) to calculate the probability of a given patient being an addict or becoming an addict. This probability score refers to the likelihood (or probability) of the patient developing OUD (opioid use disorder).

Actions (or Long-Term Analytics and Profiling:) The overall long-term goal from these analytics is to provide a recommendation to prescribers and subscribers for each patient being analyzed/reviewed for opioid prescription. The analytics, over a period of time, can be enhanced to profile prescribers and subscribers and their behavior as well.

Predictive and prescriptive analytics, when applied over an extended period, can be used to profile practitioners/prescribers and analyze their prescribing behavior by leveraging historical prescribing patterns, patient outcomes, and adherence to best practices. By continuously collecting and analyzing data on how individual practitioners prescribe medications, adjust dosages, recommend alternative treatments, and respond to patient risk scores, the system can identify trends, deviations, and potential areas for improvement. For instance, patterns of opioid prescription frequency, dosage adjustments, and responses to system-generated treatment recommendations can be evaluated to determine whether a practitioner follows evidence-based guidelines or exhibits potentially risky prescribing behavior, such as excessive opioid prescriptions or failure to modify treatment for high-risk patients. Furthermore, integration with patient outcomes can provide feedback on the effectiveness of each practitioner's treatment decisions, enabling refinement of best practices and personalized recommendations for prescribers. This profiling capability can be used to generate individualized insights for practitioners, alerting them to prescribing patterns that may require modification, while also supporting regulatory compliance and risk mitigation efforts. Additionally, aggregated profiling across multiple practitioners can assist healthcare organizations and regulatory bodies in identifying systemic issues or regional prescribing trends that may necessitate intervention, policy changes, or targeted education programs.

Similarly, the system may also be used for profiling subscribers of a drug using prescriptive analytics and predictive scores, which involves analyzing patterns of drug usage, including the quantity prescribed, strength of the drug, the frequency of prescriptions, the prescribing healthcare provider, and the locations where prescriptions are filled. This profiling also tracks how often the drug is being used, and for what indications or conditions, allowing for the identification of potential overuse, misuse, or irregular prescribing behaviors. By examining these factors, prescriptive analytics can help uncover whether subscribers are obtaining the drug from multiple sources, and if there are patterns of escalating dosages or frequent refills, which might indicate a need for closer monitoring or intervention. This profiling process, powered by predictive models, helps clinicians make informed decisions about adjusting treatment plans, switching medications, or recommending additional monitoring or therapies to ensure patient safety and improve health outcomes.

Referring and summarizing the process in FIG. 8:
1. Source Data: This is the first stage in this process, which summarizes and derives a patient's data that can be used in unsupervised predictive modeling. This data derivation is completed from the raw patient data. In this stage, various methods are used to assure that the derived attributes fit in the logistic model to provide better prediction.
2. Supervised Data Analytics (Logistic Regression): Once the explanatory variables are derived from the data, the program is run through the logistic regression.
3. Clinical Review: After generating the probability score, clinical experts review the cases to validate the classification to prepare a trained dataset to feed into the supervised learning.
4. Supervised Deep Learning: AI algorithms are used to further refine and translate the probability score into a confidence indicator to flag if the patient is likely to be readmitted within the next 30 days. The AI algorithms are trained by a large, supervised data set. The supervised data set is prepared after reviewing hundreds of cases and their subjective analysis.
5. Risk Identification: The AI algorithms provide real-time scoring based on clinical review and classification. The algorithm is implemented with a high degree of sensitivity and specificity.
6. Actions: The risk identification process will allow physicians to make more informed decisions prior to patient discharge with the goal of reducing patient readmissions within 30 days.

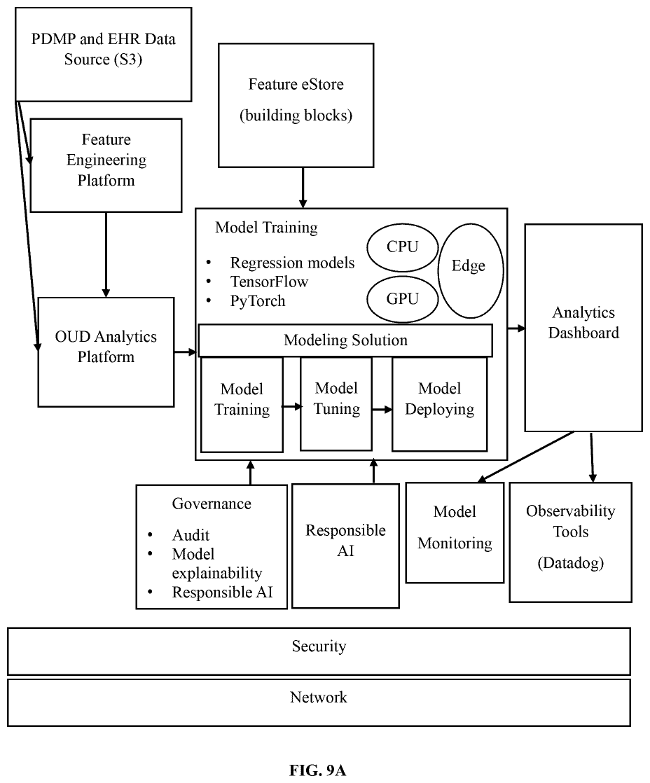

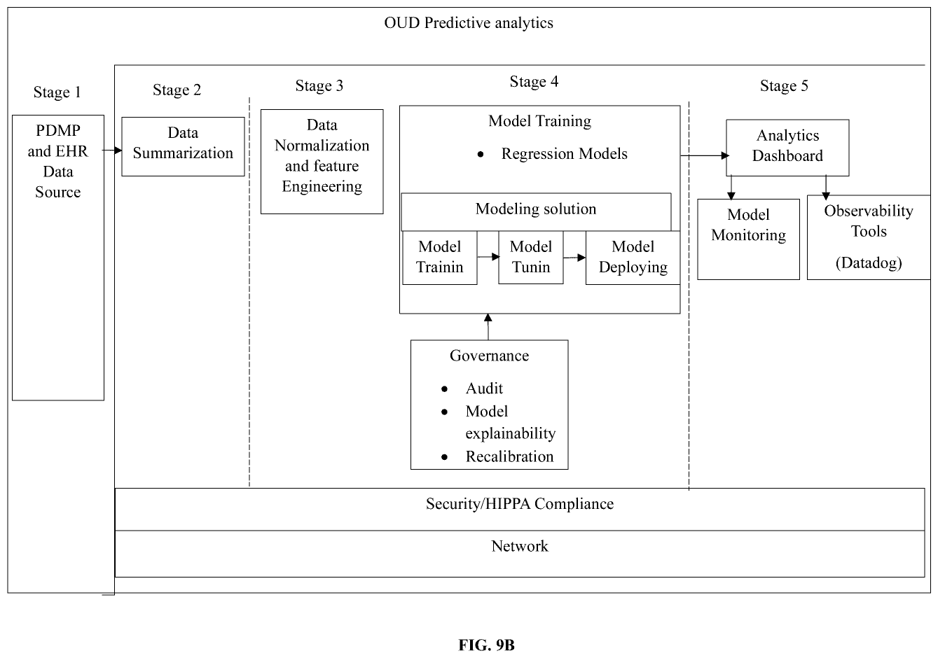

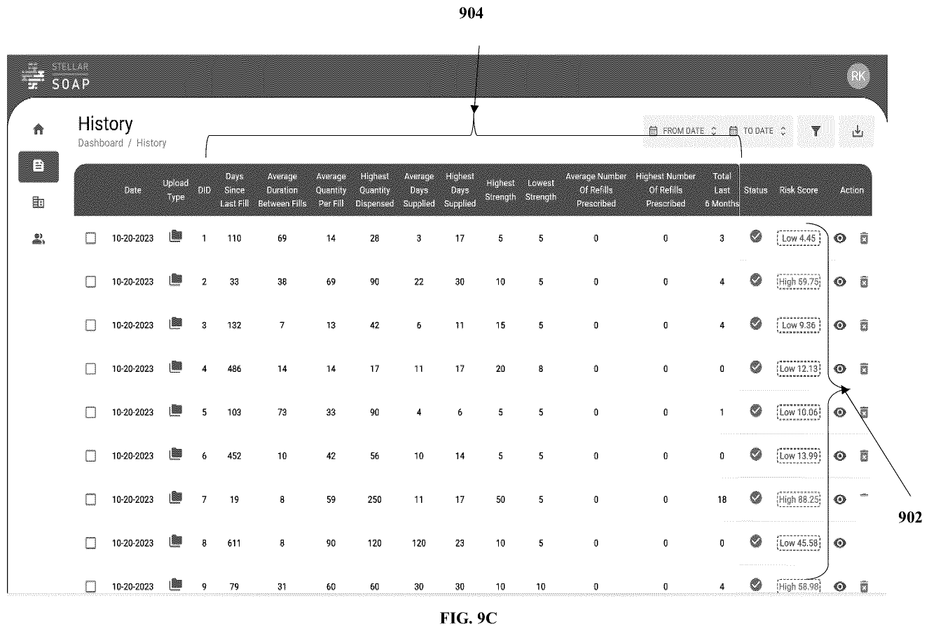

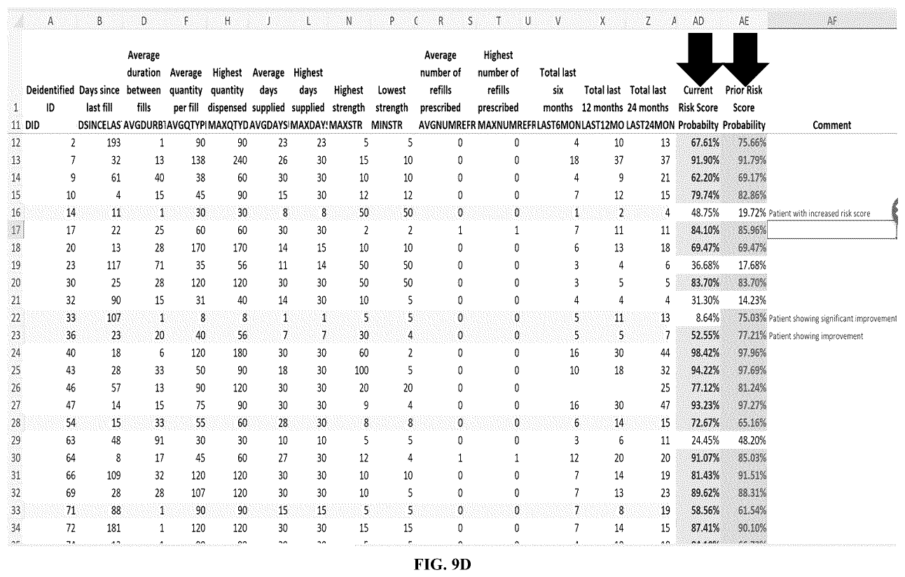

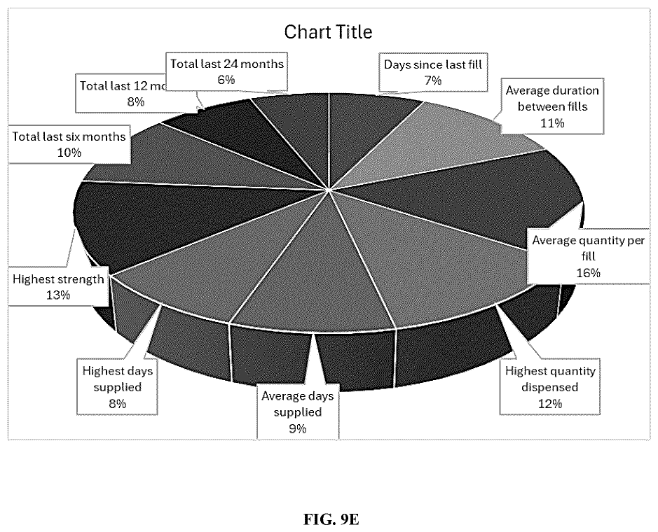

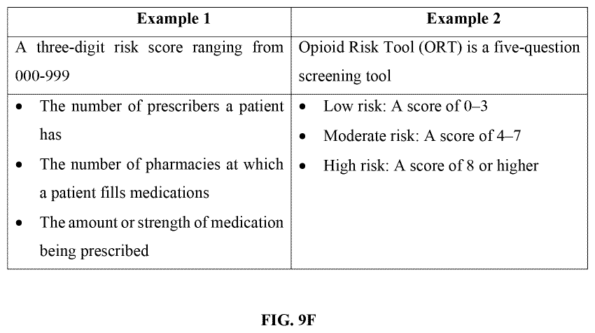

FIG. 9A illustrates Opioid Use Disorder (OUD) Architecture for Prediction Analytics according to an embodiment. The figure illustrates an AI/ML-based analytics for predicting and preventing OUD. The workflow begins with data considered from PDMP and EHR sources that may be stored in S3, where S3 (Amazon® Simple Storage Service) is a cloud-based storage service provided by Amazon Web Services (AWS). It is designed to store and retrieve any amount of data securely, offering high scalability, durability, and availability. S3 is commonly used for data storage in machine learning, analytics, backup and recovery, and big data processing. The data is then processed through a Feature Engineering Platform to extract meaningful features. These features are stored in a Feature eStore, a Feature eStore is a centralized repository designed to store, manage, and serve machine learning (ML) features efficiently. acting as a repository of reusable building blocks for model training. Additionally, an OUD (Opioid Use Disorder) Analytics Platform is integrated to support in-depth data analysis and insights. The core of the system is the Modeling Solution, which consists of three key phases: Model Training, Model Tuning, and Model Deployment. The training process utilizes various machine learning frameworks, such as regression models, TensorFlow, and PyTorch, and can be executed on CPU, GPU, or Edge computing environments. This ensures flexibility in training models across different infrastructures. The deployed models provide outputs that can be visualized through an Analytics Dashboard, enabling stakeholders, for example, physicians, pharmacists, patients, insurers, to visualize and interpret insights effectively. To maintain robust and responsible AI practices, the system incorporates Governance and Responsible AI components, ensuring auditability, model explainability, and ethical AI deployment. Model Monitoring and Observability Tools (such as Datadog) are included to track performance, detect anomalies, and ensure system reliability. The entire architecture is safeguarded by Security and Network layers, which provide protection against potential threats and ensure smooth operation.

Prescriptive analytics provide treatment recommendations to health professionals for each patient that is being analyzed/reviewed for opioid prescriptions. The analytics, over time, is also enabled to profile health professionals and their behaviors. The tools may be configured to provide predictive data to pharmacists, healthcare coordinators, insurance companies, and governmental agencies to mitigate problems and addiction before it becomes entrenched.

In an embodiment, accuracy and robustness of the predictive analytics is improved using PDMP and clinical data from multiple geographical locations. Patient numbers may be expanded to at least 50,000 for training with at least 10% reserved for performance testing and cross-validation, targeting an 85% accuracy. The data pipeline is made more robust, being able to use incomplete, non-standardized data with no reduction in accuracy.

In an embodiment, a prescriptive analytic model is created that recommends clinical interventions. Clinical recommendations from the 50,000 patients may be used to train a new model to prescribe or recommend the correct course of clinical intervention. A split of 4/96% of data may be used for training and validation, respectively. A target of a correct intervention recommendation in >90% of cases is aimed at the model.

In an embodiment, PDMP and Electronic Health Record (EHR)/Electronic medical record (EMR) are created with Direct Interface Capability. Paramount to product-market fit, the outputs within the PDMP system and EHRs are presented. Application Programming Interface (APIs) may be created for PDMP systems from at least 5 states and the 2 most widely used EHR systems, EPIC and Cerner.

### Insufficient, Reactive Approaches to OUD Identification

The sources supporting certain data and statements presented in the next three paragraphs are listed in the references section.

The increased incidence of OUD and deaths due to opioid overdose has garnered an immense amount of attention from the public resulting in bipartisan efforts within multiple levels of governing bodies producing legislation to combat the opioid. For example, in 2017 the U.S. Department of Health and Human Services launched its 5-point Opioid Strategy, allocating nearly $2 billion for the next fiscal year. By April 2018, more than half of states had established guidelines and policies to limit the amount of opioids a doctor can prescribe. A pervasive tactic for combating controlled substance misuse, OUD, and diversion is the PDMP, a state-level database of prescriptions for controlled substances. PDMPs gather prescription data, and their focus is patient-centric, intending to facilitate and encourage the identification and treatment of persons who have become addicted to prescription drugs.

However, this approach is reactive rather than proactive and only identifies those with OUD after they develop it. At this point, the cost of care to mitigate OUD is already incurred and for the rest of their life, the patient will have to combat cravings and avoid situations that, while benign to most, could induce a relapse The overall effectiveness of PDMPs as a proactive opioid risk mitigation tool is a highly debated topic with evidence being mixed and inconclusive.²⁸ For example, the House Energy and Commerce Committee stated, “The effectiveness of PDMPs is constrained by the lack of consistent utilization, timely data . . . and limited interoperability with other PDMPs.” Pharmacy lock-in programs (aka Patient Review and Restriction Programs) require patients to receive opioid prescriptions from a single pharmacy which is easily circumvented by a highly motivated individual with OUD. There is limited evidence that lock-in programs lower rates of controlled substance misuse, OUD, and diversion. For example, North Carolina Medicaid's lock-in program from 2009 to 2012 exhibited a four-fold increase in out-of-pocket controlled substance prescription fills for those enrolled in the program.

Pharmacy benefit coordinators (PBMs) are contracted by health plans, private insurers, and the government as third-party administrators of prescription drug benefit programs. PBMs inform pharmacies about which medications are covered by a drug benefit plan, manage pharmacy networks included in a plan, and process prescription drug claims. As the opioid crisis escalated, PBMs developed opioid risk management programs to detect and intervene in unsafe prescribing. As of 2018, OptumRx, Express Scripts, and CVS Caremark were included in a nationwide lawsuit initiated for their role in contributing to the opioid crisis through prescription opioid access practices, resulting in a multi-billion-dollar settlement. For now, physicians have remained relatively unscathed, though a handful of extreme circumstances resulted in the imprisonment of physicians, and the supreme court recently unanimously ruled that physicians who “knowingly mis prescribe opioids” are legally liable both in criminal and civil court.

Overall, although there are regulations, programs, and systems in place, they fail to comprehensively address the entire opioid prescription chain, focusing on single nodes rather than the system as a whole. Most importantly, the existing solutions are reactive rather than preventing OUD, a disorder patients will live with for the rest of their life.

SOLUTION: The disclosure discloses a predictive intelligent solution that can identify the patients that are likely to develop opioid use disorder.

This solution harnesses predictive and prescriptive analytics using artificial intelligence algorithms to generate an indicator (a probability score) of a patient developing OUD. Once a potential addict is identified, prescriptive analytics, which is built using machine learning models, provides treatment recommendations to assist health professionals in making more informed decisions related to drug dosage and overall approach to treatment. The prescriptive analytics then utilizes past behavior of physicians that were successful in preventing OUD or treating OUD to suggest potential courses of intervention for physicians to evaluate. Ultimately, the final intervention course lies with the physician and their expertise, but the model aims to arm them with quantitative and data-driven insights to maximize their chances of success and draw from the collective experience of the physician community rather than their individual experience and training.

The model can be used in the healthcare industry to identify and predict whether patients have become or have the potential to become abusers or addicts of commonly prescribed opioid medications. With 91% correct classification, this model had a 95% sensitivity and 96% specificity. The method to accomplish the study is outlined below.

An objective is to prospectively use the parameters and predictors for OUD (prior to initiation of opioids), defined and derived from a series of statistical methods, to classify PWH who have a 91% likelihood of developing OUD.

Approach: Assuming a prevalence of 53% for chronic opioid use among the selected population of PWH, there is a need to analyze the opioid use characteristics of 1000 subjects in clinics. This allows for a 96% positive predictive value for the model. Similar analytics that were used for the PDMP database will be used to perform a prediction analysis for development of OUD among PWH in clinics in Baltimore, MD and Washington, DC and analyze the baseline attributes (outlined further below). The algorithm can correctly predict the likelihood of OUD development over the next 6 months at a probability of >90%. The parameters to analyze include:
1. Patient Data: unique ID, age, zip code
2. Prescription Data: days since last prescription, number of prescriptions until date assessed, number of prescriptions in past 12 months, number of prescriptions in past 24 months, average duration between prescriptions
3. Drug Data: average drug quantity, highest drug quantity, average days of supply, highest days of supply, highest drug strength, lowest drug strength
4. Dispenser Data: highest number of prescriptions filled by dispenser (DEA)
5. Prescriber Data: highest number of prescriptions prescribed by prescriber for each of the parameters, find the correlation with the hypothetical outcome, and look at the distribution of each of the parameters. Whenever there is any parameter that is not normally distributed, a data transformation is performed to normalize the distribution. Typically, some of the methods used for transformations are z-score or log transformation.

### Overview of Analytics

Predictive analytics is used to develop a solution that uses a probabilistic model to generate an indicator (or probability score) that can identify a patient developing OUD. This indicator can then be used by physicians as a tool to assist in making more informed decisions related to dosage. It can also provide an opportunity to treat an at-risk patient with craving-suppression medication. The model is designed to identify those patients who carry a higher risk of addiction through a baseline probabilistic model and to constantly update the output by processing new information. The following functionality is used to generate the proposed solution:
Normalize the summarized derived patient data for a better fit in the predictive model.
Generate a probability score using the logit function.
Normalize the data distribution to a bell-shaped curve using various techniques.
Create a model, using the bell-shaped curve, with a more accurate fit and to generate a probability score.
Further refine, by calibrating the logistic equation, the probability score for the purpose of translating the score into a confidence indicator to flag the patient who is likely to become an addict.
Train the logistic model using a large, supervised data set, prepared after reviewing hundreds of cases and their subjective analysis.
Implement the AI algorithms, which provide real-time scoring based on historic claims and diagnostic data, with a high degree of sensitivity and specificity.

In addition to AI/ML algorithms, the analysis comprises a multivariate logistic regression, which comprises an analysis of heuristically selected features (explanatory/independent variables) to calculate the probability of a given patient being an addict.

FIG. 9B illustrates the OUD predictive analytics and different stages involved in predictive analytics module development and deployment according to an embodiment. The predictive analytics model is designed to identify patients who carry a higher risk of addiction. The analytics uses a probabilistic model, which uses artificial intelligence (AI) to constantly update the output by processing new information.

### Stage 1—Data Collection:

Data Sources and Processing: The probabilistic model is trained using disparate and non-standardized data sources including prescription drug monitoring programs (PDMPs) and electronic health records (EHR).

The main source of data is the PDMP (Prescription Drug Monitoring Program) and EHR (Electronic Health Records). PDMP is a prescription data collection program, particularly for opioid and other control substance drugs, that is mandated in every single state. Based on the implementation whether it is statewide, countywide or for local jurisdiction, the data is collected from the PDMP and respective EHR.

### Stage 2—Data Summarization:

Once the raw data from PDMP and EHR are collected, they are summarized or aggregated for each patient.

### Stage 3—Data Normalization and Feature Engineering:

Once the data are summarized per patient, each attribute (for the entire patient population) is analyzed to make sure their distribution is normal. For probabilistic models to work, each of the attributes should be normally distributed.

For the attributes that do not have normal distribution, a transformation function such as log, mean deviation or step function are applied. Once the data are normalized, a process called feature engineering is run to identify the attributes that are highly correlated to the dependent (or outcome) variable that is—whether a patient is an addict or not. All the attributes (that are called variables in the model) that have correlation are selected to be used in the predictive (probabilistic) model.

### Stage 4—Model Training:

Training an accurate predictive (probabilistic) model involves several steps:
1. Collecting and preparing training data
2. Designing and training a suitable model
3. Retraining over time

Data preparation involves some amount of data transformation. This preparation transforms raw data into numerical values suitable for the model. Designing and training a suitable model is an iterative, experimental process where different model structures and parameters are tested to achieve the highest accuracy. Once an initial model is trained, it can be used to make predictions on readmission. In order to improve the efficacy of the model, as well as account for changing outcomes, the model is retrained over time as new data becomes available. Over time, the model gains experience from years of data, potentially achieving very high levels of accuracy.

### Stage 5—Analytical Dashboard:

An interactive analytical dashboard summarizes data for key performance indicators as well as for better visualization. The dashboard provides updated information on a daily basis or as needed, including data in real-time. The dashboard provides reporting capabilities to monitor trends of risk scores for each patient and provides an alert for those patients who suddenly drop off from the scoring algorithm. This type of alert is designed to identify patients who are likely being refused by their health prescriber. When this occurs, it is typical for these patients to begin seeking synthetics, such as Fentanyl or Xylazine, through illicit means or the black market, which puts patients at a very high risk of experiencing an overdose. Thus, alerting the concerned authorities or physicians to such drop offs can trigger help mechanisms to check on these patients.

In the predictive analytics model, stage one is the PDMP data. All the raw data, as mentioned in FIG. 4, FIG. 5, and FIG. 6A are considered. Stage two is summarizing the data at the patient level. Then in stage three the data is normalized, wherein the data, or the attributes for all the patients combined are distributed normally, because one of the conditions for a probabilistic model is to make sure that every attribute is normally distributed, and in case the data is not normalized, then a process is run to normalize the data. After the data is normalized, the other step carried out is feature engineering. Feature engineering considers each attribute, such as the number of days since the last prescription, the number of prescriptions in the last 12 months, 24 months, etc., and the correlation in terms of whether that attribute is contributing towards the decision which is whether the patient is developing addiction or not. If there is a correlation, then those attributes are input in the model. In an example, there may be 25 attributes, but only a subset out of 25 attributes may be selected, because those are the ones contributing towards the decision-making or towards the probability of the risk score. In stage four, the identified variables or attributes are input in the probabilistic model, which is the training data set. A tuning data set is used which tunes or recalibrates the equation, and the model is then deployed. In the next stage, fifth stage, all the data is pulled in the dashboard for use by the physician, pharmacist, or by any interested, possibly authorized, party.

The data source, PDMP, includes data such as patient data, medical data, drug history, etc. The predictive data attributes as shown in FIG. 5 and FIG. 6A are used to provide prescriptive analytics which provide the treatment recommendations for the patient.

### Utility and Applications of Final Outputs:

FIG. 9C illustrates an output from the interactive dashboard from the predictive analytics module according to an embodiment. The screenshot of the dashboard shows the input data and attributes and the output probability score. Every attribute is a contributing attribute, and that is the reason it is included in the model. Some attributes may have higher weight, and some attributes may have lower weight, depending on the correlation. As seen in FIG. 9C, for certain attributes for a particular patient the value may be zero. The calculation for the weights may be done through the recalibration of the model. Recalibration may be carried out to change the weights in the model, if the result includes multiple false negatives and/or false positives. In case, for a patient record, the output is validated when it matches with that of the clinician's diagnosis. But if the clinician does not see a patient developing OUD and the output of the model is high probability of developing OUD, in that case it is a false positive. When there are multiple physician disagreements, and when such instances happen beyond a certain threshold number, which can be decided for model performance evaluation, model calibration is performed. It can happen over a period of time, for example, after a certain number of records, or while building the model, and then periodically when more and more data sets are available to validate and further train the model.

The predictive model's outcome, i.e. the risk score 902 shown for each patient and the contributing attribute or explanatory variable 904 is deployed through an interactive dashboard that summarizes data for key performance indicators as well as visualization. The dashboard provides updated information on a daily basis. The dashboard also provides reporting capabilities to monitor trends of risk scores for each patient and provides an alert for those patients who suddenly drop off from the scoring algorithm. This type of alert is designed to identify patients who are likely being refused by their health prescriber. When this occurs, it is typical for these patients to begin seeking synthetics, such as Fentanyl or Xylazine, through illicit means or the black market, which puts patients at a very high risk of experiencing an overdose.

### Calculating Predictive Risk Score and Preventing OUD:

After the predictive (probabilistic) model was developed, it was implemented to test the risk score (probability of developing OUD) on 2,000 patients. FIG. 9D shows a list of some of the patients with their individual risk score as per predictive analysis according to an embodiment. It has listed all the attributes (variables) that are used in calculating the risk score. The column ‘AE’ shows the risk score (as a percentage probability) when the model was run for the first time. As intended, the physicians may use this score and make adjustments in the prescription. Column ‘AD’ shows the new risk score for each patient calculated after a period of 6 months. It shows that the patients listed in row 22 had their risk score significantly reduced and were no longer at risk of developing OUD. This helps the physician learn that the changes made in the prescription worked and could apply to other similar patients.

Similarly, the patient listed in row 23 had the score reduced from high risk to moderate or borderline risk score. On the other hand, the patient listed in row 16 had the risk score increased to nearly developing OUD. This provides intel to the physician to look and adjust before writing a new prescription.

Overall, it is important to determine the effectiveness of any method used to tackle the OUD epidemic. Through the dashboard, healthcare agencies and physicians are able to view trend analytics to identify patients whose risk scores are decreasing month over month due to effective medical intervention, as well as tracking the patients who have a high likelihood of accessing opioids and synthetics through the black market. This type of intelligence can assist both social services and law enforcement in determining how to assist these at-risk patients.

The interactive dashboard provides the following functionalities:
Ability to easily integrate with data sources, such as PDMP and EHR.
Ability to ingest external data sources.
Ability to search for high-risk patients to provide early treatment intervention or to administer alternative medication.
Ability to monitor trends to identify patients who are at risk of switching to non-prescription off-market synthetic drugs, leading to potential overdose death.

In an embodiment, the system can track risk score over time for assessing treatment recommendation effectiveness. The system evaluates and stores, in a time series data of the risk score along with time stamp, in a database. The risk score, along with timestamped treatment interventions and adherence data, enables longitudinal monitoring. The system may further generate a visual risk progression report for healthcare providers, displaying risk score trends, treatment impact analysis, and suggest further intervention strategies to optimize patient care.

FIG. 9E illustrates the percentage of contribution of attribute variables to the risk score for the patient according to an embodiment. Disclosed technological solution uses predictive analytics to identify patients who carry the risk of developing OUD along with their contributing attributes. Once potential addiction and an addict are identified along with contributing factors for such addiction, prescriptive analytics, which is built using machine learning models, provides treatment recommendations to assist health professionals in making more informed decisions related to drug dosage and overall approach to treatment. In the current approach, using predictive analytics is unique, unlike other prior art models, the current model is explainable in the sense the model/method/system can exactly pinpoint to one or two (or as desired) top contributing risk factors that contributed to higher probability of a patient developing OUD. This probabilistic modeling makes the current approach unique and allows to provide unique and more accurate prescriptive analytics and treatment recommendations.

FIG. 9F shows examples of derivation of descriptive risk scores according to an embodiment. In an example, the Opioid Risk Tool provides a risk score between 1-10 for informing about the risk and stratify them into low, moderate, and high-risk categories as shown in column 2. In another embodiment, the risk score could be a three-digit code, where each digit represents the information as provided in column 1, first digit corresponding to the number of prescribers a patient has, the number of pharmacies at which the patient fills medications, and the amount or strength of the medication being prescribed. Other variations of representing a risk score are well contemplated.

Another objective is to develop a unique predictive algorithm for retention in case of Primary care in People With Human Immunodeficiency Virus (PWH) using a similar approach as described above.

Using the same mechanism for algorithm development as described above, a prediction model is created for retention in Human Immunodeficiency Virus (HIV) care for the next 6 months in a sample of 1000 PWH. The outline for the development and implementation of the algorithm is similar to the algorithm that was using the PDMP derived attributes.

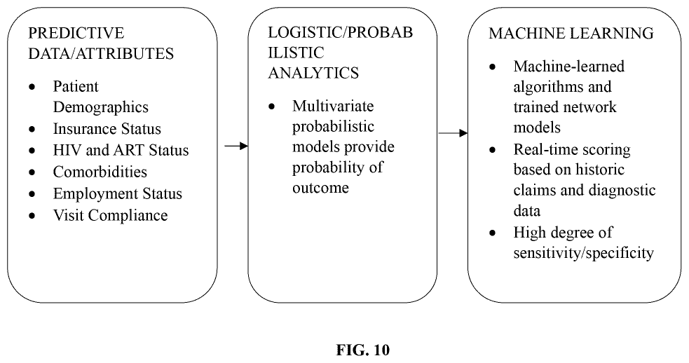

FIG. 10 illustrates methodology for algorithm development for retention in care of PWH according to an embodiment.

Predictive Data/Attributes: This includes various patient-related factors such as demographics, insurance status, HIV and ART (antiretroviral therapy) status, comorbidities, employment status, and visit compliance. These attributes serve as input features for the predictive model.

Logistic/Probabilistic Analytics: Multivariate probabilistic models analyze these attributes to estimate the probability of specific health outcomes. This step applies statistical techniques to assess risks and predict patient conditions.

Machine Learning: Advanced machine learning algorithms and trained network models to refine the predictions. Real-time scoring is performed based on historic claims and diagnostic data, ensuring high sensitivity and specificity in the predictions.

FIG. 10 represents a structured approach to leveraging the predictive and prescriptive analytics for risk prediction and decision support for retention in care of Primary care in People With Human Immunodeficiency Virus (PWH).

Data Summarization and Derivation: Logit models are used to characterize a response variable that represents a probability-like score of the target activity. The solution is implemented by applying the logit distribution to a linear, weighted combination of features to produce an estimated probability between 0 and 1. The weights of the equations are trained and recalibrated using statistical regression over a selected training set. The process to develop and implement a probabilistic score has two phases as explained herein elsewhere.

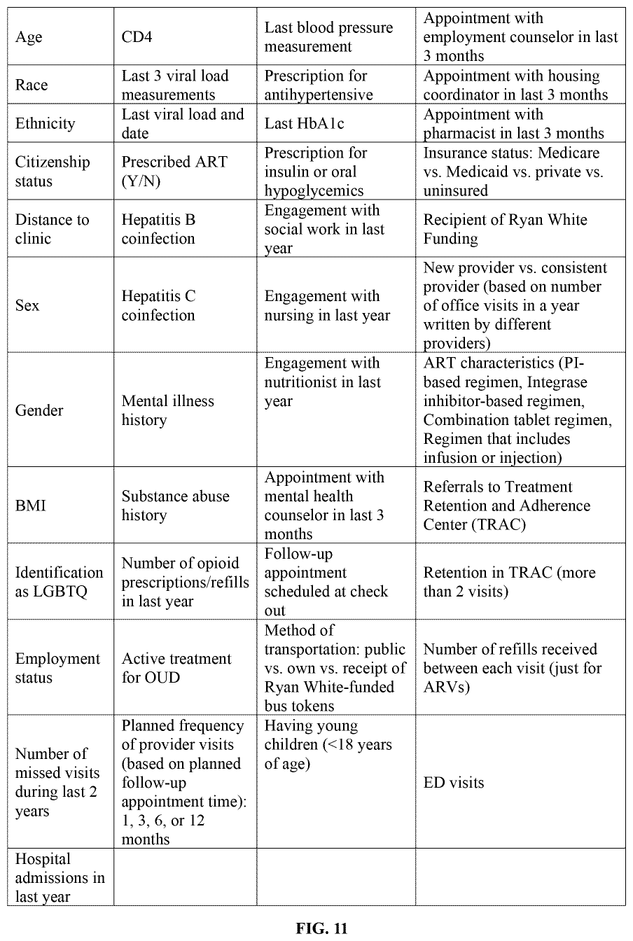

FIG. 11 illustrates data/attributes used in predictive analytics for retention in care of Primary care in people with Human Immunodeficiency Virus (PWH) according to an embodiment. Developed models can have a positive predictive value of >90% for determination of retention in care. FIG. 11 lists the attributes for analysis for an application of the model used for the HIV patient group to identify the patients at the risk of developing addiction to the prescribed drugs. The model was used on 2000 HIV patients, with the University of Maryland School of medicine, who were at the risk of developing addiction. The model was applied, and the patients were monitored over time by considering their risk score, or the probability score changing every time the prescription was changed. In some cases, the score increased, but in many cases, the probability score or risk score decreased because physicians were making the appropriate adjustments based on the risk scores of the patients.

So, for example, for one of the prescriptive drugs, a patient is getting addicted. In that case, the explanatory variables are ranked accordingly, which could be the number of times of prescribing the drug, or because of the drug strength. Then the model provides treatment recommendations using prescriptive analytics. In an embodiment, the AI/ML model used for treatment recommendations may use the clustering algorithm.

Another objective is to validate non-retention in care algorithms for retention in care. In order to prospectively validate the model in cohort, the algorithm is implemented with the ability to modify the algorithm in real-time based on analysis of probabilistic scoring and review by clinic experts, as outlined below. The model is implemented in clinics in Baltimore and Washington DC, with the calibration of the developed algorithm with a Positive Predictive Value (PPV) of >90% for determination of retention in care over the next 6 months.

Probabilistic Scoring Statistical analysis consists of a multivariate logistic regression which comprises an analysis of heuristically selected features (explanatory/independent variables) for calculation. A Θ (Theta) equation is contrived to produce a value used for calculating the logistic score. This equation is of the form Θ_{i,j,k}=C0+Σ1nCi*Fi as is described above. This probability score is then used to conduct a clinical review and prepare a test dataset to then train the AI model to produce more accurate results for each patient.

### Model Recalibration and Improvements:

In order to improve the quality and maintain consistency with the changing dynamics of patients, their disease, and their health conditions, selected cases with high probability and cases with near 50% probability can be validated on a weekly basis by a core team of clinical experts, including Infectious Disease fellows, to review these selected cases to classify them correctly in order to then prepare a supervised trained dataset to recalibrate the coefficients of each Θ equation. The same dataset can then be used to train the AI algorithms to fill the gap and increase accuracy by providing subjective analysis that could not be addressed in the probabilistic model.

Another objective is that the derived algorithm aids in improving the clinic's infrastructure for health care delivery. This is aided by long-term analytics and profiling of the algorithm, which enhances the clinic infrastructure and providers' behavior.

After the algorithm implementation and changes to clinic infrastructure, the following parameters are evaluated and the results are compared to the results prior to implementation (i.e. baseline, prior to intervention compared to 6 months after algorithm implementation):
Number of patients actively retained in THRIVE clinic care (Thrive Clinical Care or THRIVE programs, typically emphasizes holistic, patient-centered care that goes beyond disease management to promote long-term health, resilience, and quality of life), defined by those who had an in-person patient visit at least twice in twelve months
Number of patients with CD4 count (CD4 count refers to the number of CD4 T lymphocytes per cubic millimeter of blood and serves as a critical marker of immune system function, particularly in the management of HIV. CD4 cells are also known as helper T cells)>200 (CD4 count is a critical marker used in the management of HIV (Human Immunodeficiency Virus).
Number of patients with viral suppression
Number of patients on Antiretroviral Therapy (ART)
Number of patients with active insurance plan (private or public)
Number of patients engaged in Treatment Retention and Adherence Center
Number of missed visits during last year
Employment status
Number of patients screened for Hepatitis B Virus (HBV)
Number of patients screened for Hepatitis C Virus (HCV)
Proportion of patients screened determined to have chronic HCV
Proportion of patients screened determined to have active HBV
Proportion of patients found to be hepatitis B non-immune
Number of patients linked to care and evaluated for HBV treatment
Number of patients who receive HBV vaccination
Number of patients linked to care and evaluated for HCV treatment
Number of deaths
Number of deaths from drug overdose
Number of patients with HCV re-infection
Number of patients retained in Medication-Assisted Treatment (MAT) programs

### How a Technical Solution is a Technological Advancement:

In this unique approach to predictive analytics, using explainable AI, for example, using a logistic model, provides insight into specific risk factors that contribute towards a higher risk score for each patient.

Leveraging the specific knowledge about the patients and the underlying attributes (or risk factors), using artificial intelligence and machine learning algorithms to accelerate and improve the outcome of clinical trials.

Using recursive partitioning and/or random forest clustering and classification methods, the algorithm creates cohorts of high-risk patients with similar underlying risk factors. Once these cohorts are created, prescriptive analytics provides treatment recommendations and various methods of early intervention. The clustering identifies a group of patients for i) for the treatment and ii) for validation of the treatment.

This methodology and approach brings treatments to patients faster, it provides un-matched granularity and actionable insights with predictive scenarios analysis that reduces the timelines for clinical trials. It prevents enrollment delays, helps enroll more diverse patients and ensure success of the clinical program. Analytics also helps, with a high confidence level, in demonstrating a statistical significance or lack thereof in proving the effect of treatment.

### Variations or Embodiments in Artificial Intelligence Models:

In an embodiment, the AI model used for ascertaining predictive analytics may use Bayesian Network. The Bayesian network uses probability theory for prediction and provides decision-making under uncertain conditions such as the ones that may arise during the course of identifying patients and providing treatment recommendations. The Bayesian network uses nodes and arcs as in the decision trees algorithm, for providing predictive analytics and identifying patterns thereby improving clinical trials on OUD patients using predictive analytics.

In an embodiment, the AI model may use a sparse neural network for predictive and prescriptive analytics. As this model focuses on the most relevant parameters and components, it provides for reduced computations and less memory usage while the efficiency is enhanced. By using this model, the risk factors that mainly affect the outcome could be considered. Sparse models may use lasso technique for identification of the relevant features that can impact the outcome.

Techniques such as dropout and regularization can be utilized to reduce the bias in the model's prediction and increase its capability to generalize knowledge from the various characteristics to predict patient identification and provide treatment recommendations. Further, the model's hyper parameters such as depth of the network, dimensions, learning rate and momentum can be fine-tuned to improve the power of predictability of an individualized patient prediction and prescription system by leveraging the optimization techniques including, but not limited to, gradient descent, stochastic gradient descent, and their flavors (speed, memory, noise).

Risk Stratification and Insight Delivery: To increase the viability of predictive and prescriptive analytics, the prediction results can be stratified into Low, Medium, and High. This is done by the system by using modules of statistical techniques to perform operations such as normalization, standardization of predicted values and identification of thresholds to classify the prediction probability as low, medium, and high. Such a classification, in various embodiments, can help in easy assessment and interpretation of the patient prediction and prescription analytics.

Feedback layer: In addition, the system also provides its algorithms with the self-learning capabilities to learn continuously from the data provided. Such an ability in various embodiments can be potentially useful in identifying patients who are at the risk of developing OUD and prescriptive analytics for providing treatment recommendations. The feedback layer provides continuous feedback to the model making it possible to self-learn.

The system includes in various embodiments an AI suite which executes multiple machine learning models to find the optimum model yielding highest metrics of evaluation. The AI suite includes, but is not limited to, models as simple as logistic regression, and Support Vector Machine (SVM) regression to complex models such as neural networks including, but is not limited to, convolutional neural network (CNN), recurrent neural network (RNN) and long short-term memory model (LSTM).

In one possible configuration of the system, all available types of preprocessed input data can be used to train multiple models, and the best model could be used for patient prediction and prescription analytics for preventing OUD.

In another possible configuration of the system, multiple machine learning models could be trained on a subset of data, and the best ensemble of those models could be used for the prediction in various embodiments of the claimed disclosure. For example, CNN models could be trained using image scans data, RNN models could be trained using clinical trial data and medical history data and so on. Then best performing models from each input data type could be assessed for concordance among them and then all those models could be ensembled or stacked together for patient prediction and prescription analytics for preventing OUD.

The system not only uses the structured data fields like age and race but may also use unstructured data from sources like patient reports, audio, and video files of patient encounters. It may use a comprehensive list of data fields which include demographic information, clinical data (e.g., patient history), laboratory tests, investigational biomarkers, genetic testing, microbiome, imaging studies (esp. ultrasound), medications, clinical notes—by physicians, nurses, site staff, nutritional data, patient experience scores, institutional data—to investigate the impact of practice patterns, physician data (for examining the impact of individual providers on outcome) and audio/video files of Clinician-patient Interactions. The models leverage advanced computing capabilities and are not limited to: Artificial Intelligence (including neural networks, Natural Language Processing and understanding, deep learning) and traditional statistical techniques; and can analyze structured and unstructured datasets including, but not limited to: biomarkers and biochemistry data, images, genetics, clinician notes, audios and videos, demographic and socio-economic data, clinical trial data and scientific publications data. The system may continuously receive real-time feedback and accordingly improvise patient prediction and prescription analytics for OUD prevention on a perpetual basis. It leverages cutting-edge computing capabilities of AI, Mathematics and Statistics, analyzes relevant data (e.g., genetics, images, clinician's notes, audio and videos, healthcare records, wearable devices, pathology etc.) and generates unparalleled results in patient prediction and prescription analytics for preventing OUD.

The system leverages a suite of Machine/Deep Learning algorithms for exploration of factors associated with patient prediction and prescription analytics. The system adopts and stacks numerous techniques for performing tasks such as preprocessing, exploratory analysis and prediction and prescription analytics. To get a predictive model which is independent from an event, either historic or current, the model extends to include datasets, and the form of the model takes on a temporal predictive nature, with minor corrections to be made as data feedback is fed back into the model as the prediction and prescription analytics proceeds. The data used may reflect historical data-data that originated from events that were carried out or observed sometime in the past.

In an example, the factors that prove to be influential and predictive in nature to the prediction and prescription analytics, over time, with AI, get better in terms of predicting the model. The factors may change over time and the trends may change in terms of increasing issues that impinge on thresholds. The system 100 adapts as new information emerges. Machine learning in AI simplifies the process of defining future models while continuously evolving to support the selection and identification of clinical trial data and publications data with a high probability of identifying patients who are at the risk of developing OUD and performing prescriptive analytics for providing treatment recommendations.

The system uses AI applications like Natural Language Processing (NLP) techniques that analyze unstructured data, such as medical literature and adverse event reports, to extract meaningful insights related to drug interactions, adverse effects, or patient outcomes. Machine Learning (ML) algorithms, including supervised learning classification algorithms, aid in categorizing treatment recommendations based on historical patterns and existing labeled data. This analysis helps in identifying potential risks by extracting meaningful insights from the data.

In an embodiment, the system may use AI-driven anomaly detection techniques such as machine learning algorithms like Isolation Forests or One-Class SVM that detect unusual data patterns or outliers that may identify patients and provide treatment recommendations. As an example, the classification is based on whether the patient identification or treatment recommendation is at or above the threshold level, and the data used to make the classification (prediction) consist of the remaining acquired data as a function of time. SVM could be trained to predict patient identification and treatment recommendation from the other coincident data, and/or could be trained to predict from the other data whether the probability of patient identification can be higher in a similar setting. The ML model may further utilize clustering techniques, where the machine learning algorithm is provided with unlabeled or unclassified data, which leaves the algorithm to identify hidden structures amongst the cases. SVM can be used by a user interface to perform SVM training, classification, and prediction, and to automatically capture and identify clusters and generate output to a user interface. The Classifier model could be a cross-platform user interface. In SVM clustering, the clustering can be further combined with k-means clustering to accelerate the training and prediction of the model.

In an embodiment, the AI model used for the predictive and prescriptive analytics comprises an Explainable artificial intelligence (XAI) model or Explainable AI model. Explainable AI could be used to describe an AI model that provides reasoning or justification for the decisions taken by the model thus allowing for transparency in the model. XAI algorithms are programmed to describe its purpose, rationale and decision-making process in a way that can be understood by the average person. Explainable AI may address certain issues pertaining to AI-based analysis such as bias, transparency, safety, and causality. Bias refers to potentially flawed AI resulting from biased training data. xAI is often discussed in relation to deep learning and plays a key role in the FAT ML model (fairness, accountability, and transparency in machine learning). As the model provides insights into taking its decisions and making predictions, the results of the analytics by this model is better understood by the users of the model. Explainable AI technique provides an effective manner of illustrating the back-end reasoning process of the AI system at a considerably granular level, which allows the user to make an informed decision or accept a decision made on behalf thereof.

As the model allows for understanding the various features, the model may determine the keywords which need more weightage. The disclosure provides an individualized patient prediction and prescription model based on a configurable system of weightings assigned to the keywords. The model also provides for model explainability so that the user understands the model, how the end result is reached, thereby resulting in more acceptance of the model.

Data Enrichment and Model Refinement could be performed to expand the dataset and refine predictive models with new inputs. Environmental and behavioral data is integrated, and the model is configured for continuous Learning Mechanism. When combined into a single system, detection and prediction models create a synergistic effect that offers a comprehensive approach for an individualized patient prediction and prescription system.

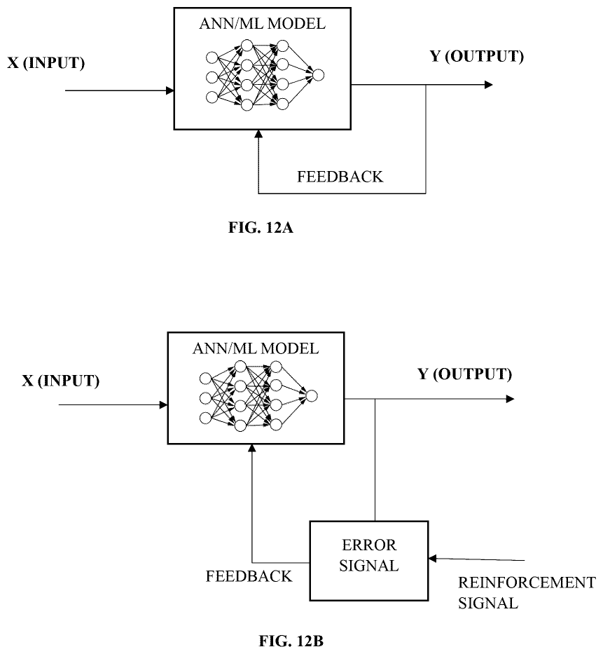

FIG. 12A shows a structure of the neural network/machine learning model with a feedback loop. An Artificial neural networks (ANNs) model comprises an input layer, one or more hidden layers, and an output layer. Each node, or artificial neuron, connects to another and has an associated weight and threshold. If the output of any individual node is above the specified threshold value, that node is activated, sending data to the next layer of the network. Otherwise, no data is passed to the next layer of the network. A machine learning model or an ANN model may be trained on a set of data to take a request in the form of input data, make a prediction on that input data, and then provide a response. The input to the model may include data extracted from clinical repositories and scientific publications. The model may learn from the data. Learning can be supervised learning and/or unsupervised learning and may be based on different scenarios and with different datasets. Supervised learning comprises logic using at least one of a decision tree, logistic regression, and support vector machines. Unsupervised learning comprises logic using at least one of a k-means clustering, a hierarchical clustering, a hidden Markov model, and an apriori algorithm. The output layer may predict an individualized patient prediction and prescription recommendation system based on a predefined threshold value and improve clinical trials on OUD patients using predictive analytics.

In an embodiment, ANN may be a Deep Neural Network (DNN), which is a multilayer tandem neural network comprising Artificial Neural Networks (ANN), Convolution Neural Networks (CNN) and Recurrent Neural Networks (RNN) that can recognize features from inputs, do an expert review, and perform actions that require predictions, creative thinking, and analytics. In an embodiment, ANNs may be Recurrent Neural Network (RNN), which is a type of Artificial Neural Networks (ANN), which uses sequential data or time series data. Deep learning algorithms are commonly used for ordinal or temporal problems, such as language translation, Natural Language Processing (NLP), speech recognition, and image recognition, etc. Like feedforward and convolutional neural networks (CNNs), recurrent neural networks utilize training data to learn. They are distinguished by their “memory” as they take information from prior input via a feedback loop to influence the current input and output. An output from the output layer in a neural network model is fed back to the machine learning model through the feedback. The variations of weights in the hidden layer(s) are adjusted to fit the expected outputs better while training the model. This allows the model to provide results with far fewer mistakes.

The neural network is featured with the feedback loop to adjust the system output dynamically as it learns from the new data. In machine learning, backpropagation and feedback loops are used to train an AI model and continuously improve it upon usage. As the incoming data that the model receives increases, there are more opportunities for the model to learn from the data. The feedback loops, or backpropagation algorithms, identify inconsistencies and feed the corrected information back into the model as an input.

Even though the AI/ML model is trained well, with large sets of labeled data and concepts, after a while, the models' performance may decline while adding new, unlabeled input due to many reasons which include, but not limited to, concept drift, recall precision degradation due to drifting away from true positives, and data drift over time. A feedback loop to the model keeps the AI results accurate and ensures that the model maintains its performance and improvement, even when new unlabeled data is assimilated. A feedback loop refers to the process by which an AI model's predicted output is reused to train new versions of the model. This is also used to make the model a self-learning model.

Initially, when the AI/ML model is trained, a few labeled samples comprising both positive and negative examples of the concepts (for e.g., using artificial intelligence algorithms to generate an indicator (a probability score) of a patient developing OUD) are used that are meant for the model to learn. Afterward, the model is tested using unlabeled data. By using, for example, deep learning and neural networks, the model can then make predictions on whether the desired concept/s (e.g., generation of an indicator (a probability score) of a patient developing OUD) are in unlabeled images. Each image is given a probability score where higher scores represent a higher level of confidence in the models' predictions. Where a model gives an image a high probability score, it is auto-labeled with the predicted concept. However, in the cases where the model returns a low probability score, this input may be sent to a controller (may be a human moderator) which verifies and, as necessary, corrects the result. The human moderator may be used only in exception cases. The feedback loop feeds labeled data, auto-labeled or controller-verified data back to the model dynamically and is used as training data so that the system can improve its predictions in real-time and dynamically.

FIG. 12B shows a structure of the neural network/machine learning model with reinforcement learning. The network receives feedback from authorized networked environments. Though the system is similar to supervised learning, the feedback obtained in this case is evaluative not instructive, which means there is no teacher as in supervised learning. After receiving the feedback, the network performs adjustments of the weights to get better predictions in the future. Machine learning techniques, like deep learning, allow models to take labeled training data and learn to recognize those concepts in subsequent data and images. The model may be fed with new data for testing, hence by feeding the model with data it has already predicted over, the training gets reinforced. If the machine learning model has a feedback loop, the learning is further reinforced with a reward for each true positive of the output of the system. Feedback loops ensure that AI results do not stagnate. By incorporating a feedback loop, the model output keeps improving dynamically and over usage/time. This also results in making the model a self-learning model.

In an embodiment, the machine learning model is configured to learn using labeled data using a supervised learning method, wherein the supervised learning method comprises logic using at least one of a decision tree, a logistic regression, a support vector machine, a k-nearest neighbors, a Naïve Bayes, a random forest, a linear regression, a polynomial regression, and a support vector machine for regression.

In some embodiments, the machine learning model is configured to learn from a real-time data using an unsupervised learning method, wherein the unsupervised learning method comprises logic using at least one of a k-means clustering, a hierarchical clustering, a hidden Markov model, and an apriori algorithm.

In some embodiments, the machine learning model has a feedback loop, wherein an output from a previous step is fed back to the machine learning model in real-time to improve the performance and accuracy of the output of a next step.

In some embodiments, the machine learning model has a feedback loop, wherein the learning is further reinforced with a reward for each true positive of the output of the system.

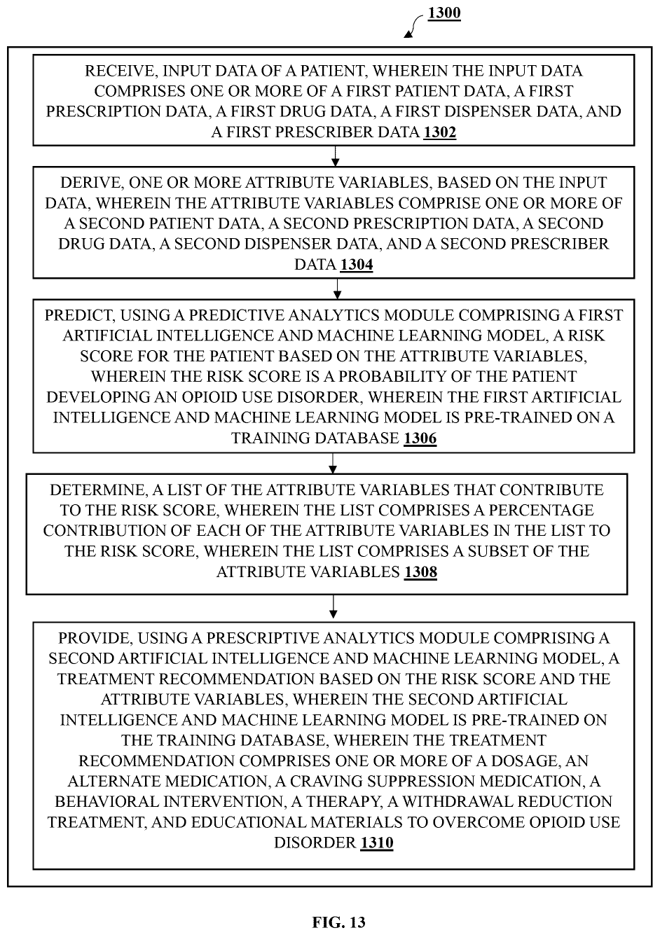

FIG. 13 illustrates a flow chart describing a method implemented in the opioid risk tool according to an embodiment.

According to an embodiment, it is a method 1300 comprising, receiving, input data of a patient, wherein the input data comprises one or more of a first patient data, a first prescription data, a first drug data, a first dispenser data, and a first prescriber data at step 1302; deriving, one or more attribute variables, based on the input data, wherein the attribute variables comprise one or more of a second patient data, a second prescription data, a second drug data, a second dispenser data, and a second prescriber data at step 1304; predicting, using a predictive analytics module comprising a first artificial intelligence and machine learning model, a risk score for the patient based on the attribute variables, wherein the risk score is a probability of the patient developing an opioid use disorder, wherein the first artificial intelligence and machine learning model that provides explainable outcome and is pre-trained on a training database at step 1306; and determining, a list of the attribute variables that contribute to the risk score, wherein the list comprises a percentage contribution of each of the attribute variables in the list to the risk score, wherein the list comprises a subset of the attribute variables at step 1308; providing, using a prescriptive analytics module comprising a second artificial intelligence and machine learning model, a treatment recommendation based on the risk score and the attribute variables, wherein the second artificial intelligence and machine learning model is pre-trained on the training database, wherein the treatment recommendation comprises one or more of a dosage, an alternate medication, and craving-suppression medication, a behavioral intervention, a therapy, a withdrawal reduction treatment, and educational materials to overcome the opioid use disorder at step 1310; and wherein the method is configured to one or more of identifying and preventing the opioid use disorder in the patient, wherein the patient is being treated for pain with a drug via prescription, and wherein the drug is an opioid.

According to an embodiment of the method, wherein the risk score along with the list of attributes is one or more of displayed on a display, stored to a database, and generates an alarm. Generating an alarm takes place when the risk score is above a certain threshold value, which may be pre-set. The pre-set limit may be decided based on physician's inputs. In an embodiment, if there is a change in the risk score in the current visit from that of a last visit of the patient to a physician, the system highlights the record and generates an alarm to bring attention to the risk of OUD for that patient. An alarm can be generated through various modes, including audible alarms (sirens, beeps, or voice alerts), visual alarms (highlights or flashing display), tactile alarms (vibrations or haptic feedback on a device), and digital notifications (text alerts, emails, or app notifications). According to an embodiment of the method, the first artificial intelligence and machine learning model and the second artificial intelligence and machine learning model are retrained based on false positive and false negative results during a period of use of the first artificial intelligence and machine learning model and the second artificial intelligence and machine learning model.

According to an embodiment, it is a method 1300 comprising, receiving, input data of a patient, wherein the input data comprises one or more of a first patient data, a first prescription data, a first drug data, a first dispenser data, and a first prescriber data at step 1302; deriving, one or more attribute variables, based on the input data, wherein the attribute variables comprise one or more of a second patient data, a second prescription data, a second drug data, a second dispenser data, and a second prescriber data at step 1304; predicting, using a predictive analytics module comprising a first artificial intelligence and machine learning model, a risk score for the patient based on the attribute variables, wherein the risk score is a probability of the patient developing an opioid use disorder, wherein the first artificial intelligence and machine learning model that provides explainable outcome and is pre-trained on a training database at step 1306; and determining, a list of the attribute variables that contribute to the risk score, wherein the list comprises a percentage contribution of each of the attribute variables in the list to the risk score, wherein the list comprises a subset of the attribute variables at step 1308; and wherein the method is configured to one or more of identifying and preventing the opioid use disorder in the patient, wherein the patient is being treated for pain with a drug via prescription, and wherein the drug is an opioid. The step of providing, using a prescriptive analytics module comprising a second artificial intelligence and machine learning model, a treatment recommendation based on the risk score and the attribute variables, wherein the second artificial intelligence and machine learning model is pre-trained on the training database, wherein the treatment recommendation comprises one or more of a dosage, an alternate medication, and craving-suppression medication, a behavioral intervention, a therapy, a withdrawal reduction treatment, and educational materials to overcome the opioid use disorder at step 1310 may be independent and a separate module.

According to an embodiment of the method, the system receives real-time patient adherence data by pharmacy refill records, dynamically updates the risk score, and modifies treatment recommendations accordingly.

According to an embodiment of the method, the first artificial intelligence and machine learning model continuously updates based on patient treatment response data, refining future risk assessments and treatment recommendations through a self-learning mechanism.

According to an embodiment of the method, upon detecting a high-risk patient attempting to fill an opioid prescription, the system generates an immediate alert to the prescribing physician and pharmacist, providing a risk summary and alternative treatment recommendations.

According to an embodiment of the method, upon detecting a high-risk patient attempting to fill an opioid prescription, the system alerts the pharmacist to stop providing the medication and generate an appointment with a physician.

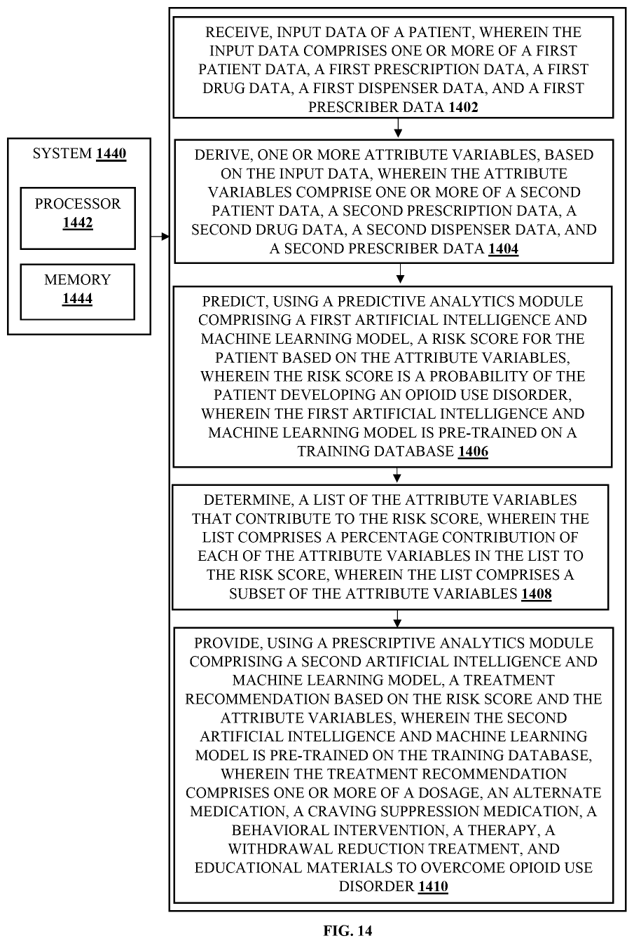

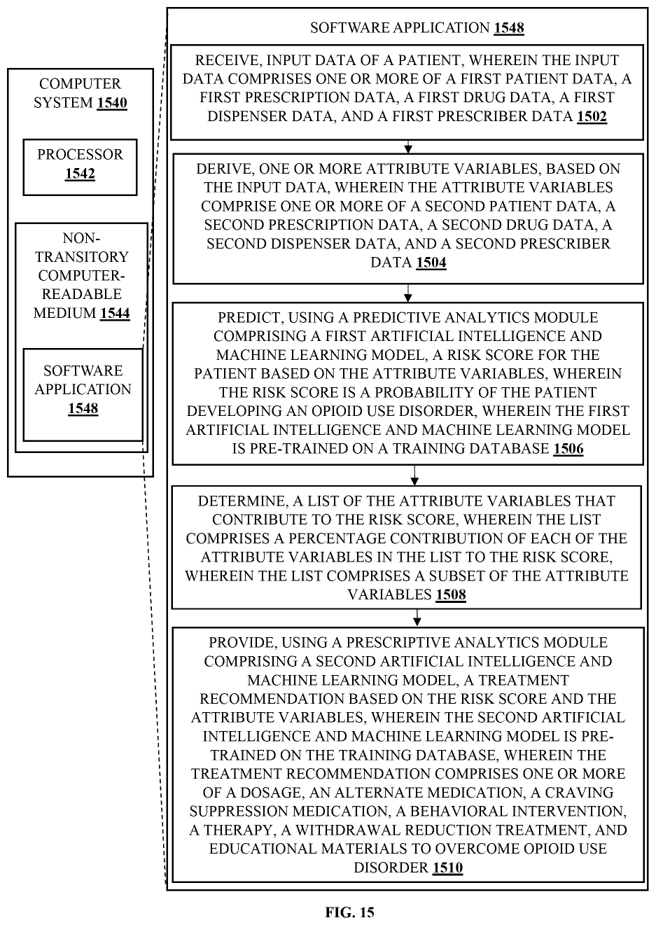

FIG. 14 illustrates a block diagram of the system implementing the method according to an embodiment. According to an embodiment, disclosed is system 1440 comprising processor 1442, and memory 1444, wherein the memory 1444 storing processor-executable instructions, which on execution, cause the processor to receive, input data of a patient, wherein the input data comprises one or more of a first patient data, a first prescription data, a first drug data, a first dispenser data, and a first prescriber data at step 1402; derive, one or more attribute variables, based on the input data, wherein the attribute variables comprise one or more of a second patient data, a second prescription data, a second drug data, a second dispenser data, and a second prescriber data at step 1404; predict, using a predictive analytics module comprising a first artificial intelligence and machine learning model, a risk score for the patient based on the attribute variables, wherein the risk score is a probability of the patient developing an opioid use disorder, wherein the first artificial intelligence and machine learning model that provides explainable outcome and is pre-trained on a training database at step 1406; and determine, a list of the attribute variables that contribute to the risk score, wherein the list comprises a percentage contribution of each of the attribute variables in the list to the risk score, wherein the list comprises a subset of the attribute variables at step 1408; provide, using a prescriptive analytics module comprising a second artificial intelligence and machine learning model, a treatment recommendation based on the risk score and the attribute variables, wherein the second artificial intelligence and machine learning model is pre-trained on the training database, wherein the treatment recommendation comprises one or more of a dosage, an alternate medication, a craving-suppression medication, a behavioral intervention, a therapy, a withdrawal reduction treatment, and educational materials to overcome the opioid use disorder at step 1410; and wherein the system is configured to one or more of identifying and preventing the opioid use disorder in the patient, wherein the patient is being treated for pain with a drug via prescription, and wherein the drug is an opioid.

According to an embodiment of the system, the input data is considered from one or more databases, wherein the databases comprise Prescription Drug Monitoring Program (PDMP) and Electronic Health Record (EHR).

According to an embodiment of the system, the system integrates with electronic health record (EHR) systems to provide real-time opioid risk assessments at the point of prescribing, thereby reducing the likelihood of overprescription and misuse.

According to an embodiment of the system, the predictive analytics module utilizes a neural network architecture configured to dynamically adjust feature weighting based on real-time prescription refill patterns.

According to an embodiment of the system, the system is configured to integrate with an electronic health record (EHR) system to automatically retrieve and analyze real-time prescription data, alerting prescribers and pharmacists if a patient exhibits high-risk opioid use patterns before medication is prescribed or dispensed.

According to an embodiment of the system, upon determining that the risk score exceeds a predetermined threshold, the system automatically modifies the prescription by reducing the dosage, suggesting a non-opioid alternative, or recommending a follow-up patient consultation.

According to an embodiment of the system, the first patient data comprises at least one or more of patient identification details, patient date of birth, and patient location. According to an embodiment of the system, the first prescription data comprises at least one or more of prescription filled data, prescription date, and prescription number. According to an embodiment of the system, the first drug data comprises at least one or more of drug name, drug strength, drug form, drug quantity, and number of days of supply. According to an embodiment of the system, the first dispenser data comprises one or more of dispenser identification details and dispenser location. According to an embodiment of the system, the first prescriber data comprises one or more of prescriber identification details and prescriber location.

According to an embodiment of the system, the second patient data comprises one or more of patient unique identification, patient age, and patient location. According to an embodiment of the system, the second prescription data comprises one or more of number of prescriptions in a time period, days since last prescription, number of prescription in a past selected period, and average duration between prescriptions. According to an embodiment of the system, the second drug data comprises one or more of average drug quantity, highest drug quantity, average days of supply, highest days of supply, highest drug strength, lowest drug strength as per the first prescription data and the first drug data. According to an embodiment of the system, the second dispenser data comprises one or more of dispenser identification details, dispenser location, highest number of prescriptions filled by a dispenser, identification details of most frequently used dispenser for filling the drug. According to an embodiment of the system, the second prescriber data comprises one or more of prescriber identification details, prescriber location, highest number of prescriptions prescribed, and most frequent prescriber identification details.

According to an embodiment of the system, the first artificial intelligence and machine learning model comprises a logistic regression model. According to an embodiment of the system, the logistic regression model applies a linear and weighted contribution of the input data; and wherein the logistic regression model comprises a multivariate logistic regression. According to an embodiment of the system, the risk score is a value between 0 and 1.

According to an embodiment of the system, the second artificial intelligence and machine learning model comprises a clustering model. According to an embodiment of the system, the clustering model comprises one of recursive partitioning and random forest clustering. According to an embodiment of the system, the clustering model groups the patient with similar underlying attributes into a cluster of the plurality of patients from training input data. According to an embodiment of the system, the training database comprises training input data from a plurality of patients. According to an embodiment of the system, the training input data is preprocessed by cleaning and normalizing the training input data.

According to an embodiment of the system, the system is configured for use by a physician as a decision support system for prescribing one or more of the prescription drug and the treatment recommendation.

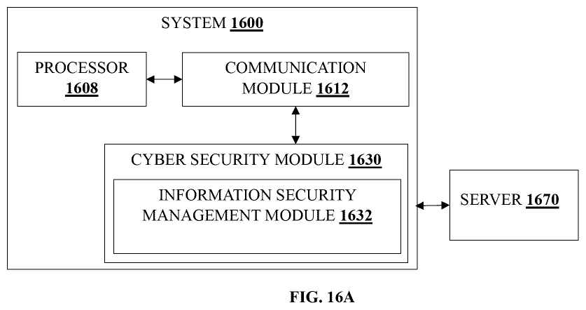

FIG. 15 illustrates a block diagram of the method executed by the non-transitory computer-readable medium according to an embodiment.

According to an embodiment, disclosed is non-transitory computer-readable medium 1544 having stored thereon instructions executable by computer system 1540 to perform operations 1548 comprising receiving, input data of a patient, wherein the input data comprises one or more of a first patient data, a first prescription data, a first drug data, a first dispenser data, and a first prescriber data at step 1502; deriving, one or more attribute variables, based on the input data, wherein the attribute variables comprise one or more of a second patient data, a second prescription data, a second drug data, a second dispenser data, and a second prescriber data at step 1504; predicting, using a predictive analytics module comprising a first artificial intelligence and machine learning model, a risk score for the patient based on the attribute variables, wherein the risk score is a probability of the patient developing an opioid use disorder, wherein the first artificial intelligence and machine learning model that provides explainable outcome and is pre-trained on a training database at step 1506; and determining, a list of the attribute variables that contribute to the risk score, wherein the list comprises a percentage contribution of each of the attribute variables in the list to the risk score, wherein the list comprises a subset of the attribute variables at step 1508; providing, using a prescriptive analytics module comprising a second artificial intelligence and machine learning model, a treatment recommendation based on the risk score and the attribute variables, wherein the second artificial intelligence and machine learning model is pre-trained on the training database, wherein the treatment recommendation comprises one or more of a dosage, an alternate medication, and craving-suppression medication, a behavioral intervention, a therapy, a withdrawal reduction treatment, and educational materials to overcome opioid use disorder at step 1510; and wherein the operations are configured to one or more of identifying and preventing opioid use disorder in the patient, wherein the patient is being treated for pain with a drug via prescription, and wherein the drug is an opioid.

According to an embodiment, it is a system comprising a processor 1542 storing instructions in a non-transitory memory that, when executed, cause the processor to acquire patient data related to patients; preprocess the patient data to obtain preprocessed patient data; train, a first machine learning model with the preprocessed patient data, wherein the first machine learning model is configured to: analyze one or more features from the preprocessed patient data of the patient; predict a risk score of the patient at risk of developing opioid use disorder; and compute prescriptive analytics to provide a treatment recommendation based on the predicted risk score; and wherein the trained first machine learning model is configured to identify patients and prevent developing opioid use disorder.

According to an embodiment of the system, the processor is configured to apply logistic distribution to a linear, weighted combination of the one or more features to produce the risk score between 0 and 1. According to an embodiment of the system, the preprocessed patient data comprises at least one of structured data, semi-structured data, and unstructured data. According to an embodiment of the system, the preprocessed patient data is stored in a database. According to an embodiment of the system, the patient data comprises at least one of a textual data, a numerical data, a graphical representation, a chart, and a table. According to an embodiment of the system, the first machine learning model is further configured to: cleanse and filter the preprocessed patient data based on at least one of an input from a user, and a predefined rule.

According to an embodiment of the system, the first machine learning model is configured to perform one or more of a normalization, a standardization, and a stratification of the preprocessed patient data. According to an embodiment of the system, the system is further configured to: customize the first machine learning model by at least one of manipulating the preprocessed patient data to obtain manipulated preprocessed patient data, adding, modifying, removing at least one node of the first machine learning model, and train the first machine learning model using the manipulated preprocessed patient data. According to an embodiment of the system, the system is further configured to enable a user to interact with a server through a user interface, provided via a device associated with the user, and at least one of build, train, retrain, replicate, compare, and share the first machine learning model.

According to an embodiment of the system, the system is configured to display the risk score. According to an embodiment of the system, the first machine learning model is calibrated and selected from one or more artificial intelligence (AI) models for computing predictive and prescriptive analytics by evaluating AI model candidates against a set of performance criteria. According to an embodiment of the system, the set of performance criteria comprise prediction accuracy, computational efficiency, and adaptability to diverse preprocessed data. According to an embodiment of the system, the first machine learning model is configured to learn using labeled data using a supervised learning model, wherein the supervised learning model comprises logic using at least one of a decision tree, a logistic regression, a support vector machine, a k-nearest neighbors, a Naïve Bayes, a random forest, a linear regression, a polynomial regression, and a support vector machine for regression.

According to an embodiment of the system, the first machine learning model has a feedback loop, wherein an output from a previous step is fed back to the model in real-time to improve performance and accuracy of the output of a next step. According to an embodiment of the system, the first machine learning model is a self-learning model. According to an embodiment of the system, the first machine learning model comprises a feedback loop, wherein the learning is further reinforced with a reward for each true positive of an output of the system. According to an embodiment of the system, the processor is further configured to assign a weight to the one or more features based on a first pattern of outcomes of historical patient data. According to an embodiment of the system, the processor is further configured to modify the weight of the one or more features based on a second pattern of outcomes of training patient data.

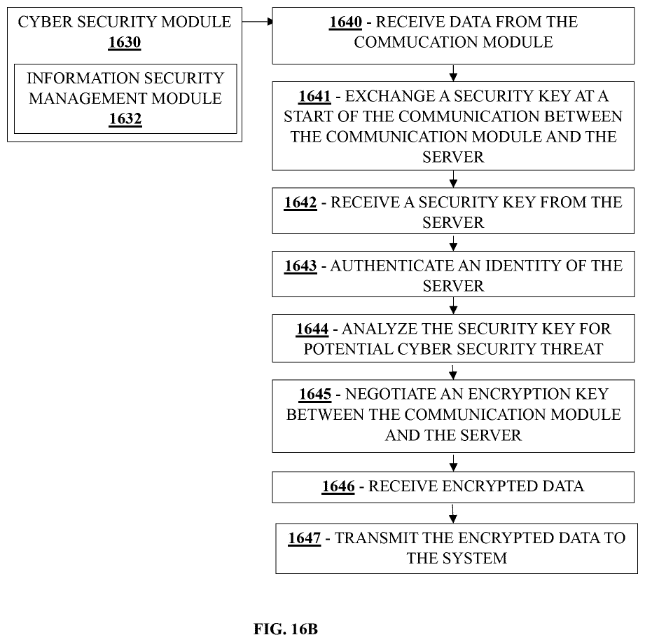

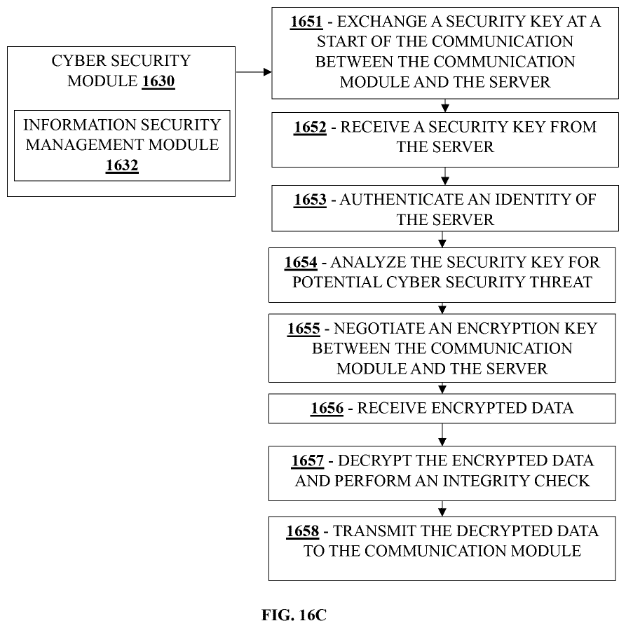

FIG. 16A illustrates the block diagram of the cyber security module in view of the system and server according to an embodiment.

In one aspect, a secure communication management (SCM) computer device for providing secure data connections is provided. The SCM computer device includes a processor in communication with memory. The processor is programmed to receive, from a first device, a first data message. The first data message is in a standardized data format. The processor is also programmed to analyze the first data message for potential cyber security threats.

According to an embodiment, secure authentication for data transmissions comprises, provisioning a hardware-based security engine (HSE) located in communications system, said HSE having been manufactured in a secure environment and certified in said secure environment as part of an approved network; performing asynchronous authentication, validation and encryption of data using said HSE, storing user permissions data and connection status data in an access control list used to define allowable data communications paths of said approved network, enabling communications of the communications system with other computing system subjects to said access control list, performing asynchronous validation and encryption of data using security engine including identifying a user device (UD) that incorporates credentials embodied in hardware using a hardware-based module provisioned with one or more security aspects for securing the system, wherein security aspects comprising said hardware-based module communicating with a user of said user device and said HSE.

Referring to FIG. 16A, system 1600 comprising processor 1608 communication module 1612, cyber security module 1630, and information security management module 1632 in communication with server 1670, is shown. In an embodiment, FIG. 16A shows the block diagram of the cyber security module. The communication of data from the processor 102, shown in FIG. 1, of the system 100 and the server 1670 through the communication module 1612 is first verified by the information security management module 1632 before being transmitted from the system to the server or from the server to the system. The information security management module is operable to analyze the data for potential cyber security threats, to encrypt the data when no cyber security threat is detected, and to transmit the data encrypted to the system or the server. In an embodiment, the communication of data from the processor 102 to the servers 110 in FIG. 1, comprises the data that is communicated by the communication module 1612 to the server 1670. The communication of data from the processor 102 to the database 108, the communication module 118 in FIG. 1, may be first verified by the information security management module 1632 of the cyber security module 1630 to ensure integrity of data. Once the data is sourced, for that data to accurately help identify patients who are at the risk of developing OUD and providing an individualized patient prediction and prescription for preventing OUD, it may be kept from unauthorized access by verification provided by the cyber security module 1630. In an embodiment, the data sourced may also be verified by the cyber security module to ensure that the data has not been tampered with. Further, as system data include data relating to patients, to ensure that the data is not compromised, the privacy and security of data may be protected by verifying the data by the cyber security module 1630.

FIG. 16B shows an embodiment of the cyber security module, in accordance with some embodiments in the present disclosure.

In an embodiment, the cyber security module further comprises an information security management module providing isolation between the system and the server. FIG. 16B shows the flowchart of securing the data through the cyber security module 1630. At step 1640, the information security management module is operable to receive data from the communication module. At step 1641, the information security management module exchanges a security key at a start of the communication between the communication module and the server. At step 1642, the information security management module receives a security key from the server. At step 1643, the information security management module authenticates an identity of the server by verifying the security key. At step 1644, the information security management module analyzes the security key for potential cyber security threats. At step 1645, the information security management module negotiates an encryption key between the communication module and the server. At step 1646, the information security management module receives the encrypted data. At step 1647, the information security management module transmits the encrypted data to the server when no cyber security threat is detected.

FIG. 16C shows another embodiment of the cyber security module, in accordance with some embodiments in the present disclosure.

In an embodiment, FIG. 16C shows the flowchart of securing the data through the cyber security module 1630. At step 1651, the information security management module 1632 is operable to: exchange a security key at a start of the communication between the communication module and the server. At step 1652, the information security management module receives a security key from the server. At step 1653, the information security management module 1632 authenticates an identity of the server by verifying the security key. At step 1654, the information security management module analyzes the security key for potential cyber security threats. At step 1655, the information security management module negotiates an encryption key between the communication module and the server. At step 1656, the information security management module receives encrypted data. At step 1657, the information security management module decrypts the encrypted data, and performs an integrity check of the decrypted data. At step 1658, the information security management module transmits the decrypted data to the communication module when no cyber security threat is detected.

In an embodiment, the integrity check is a hash-signature verification using a Secure Hash Algorithm 256 (SHA256) or a similar method.

In an embodiment, the information security management module is configured to perform asynchronous authentication and validation of the communication between the communication module and the server.

In an embodiment, the information security management module is configured to raise an alarm if a cyber security threat is detected. In an embodiment, the information security management module is configured to discard the encrypted data received if the integrity check of the encrypted data fails.

In an embodiment, the information security management module is configured to check the integrity of the decrypted data by checking accuracy, consistency, and any possible data loss during the communication through the communication module.

In an embodiment, the server is physically isolated from the system through the information security management module. When the system communicates with the server as shown in FIG. 16A, identity authentication is first carried out on the system and the server. The system is responsible for communicating/exchanging a public key of the system and a signature of the public key with the server. The public key of the system and the signature of the public key are sent to the information security management module. The information security management module decrypts the signature and verifies whether the decrypted public key is consistent with the received original public key or not. If the decrypted public key is verified, the identity authentication is passed. Similarly, the system and the server carry out identity authentication on the information security management module. After the identity authentication is passed on to the information security management module, the two communication parties, the system, and the server, negotiate an encryption key and an integrity check key for data communication of the two communication parties through the authenticated asymmetric key. A session ID number is transmitted in the identity authentication process, so that the key needs to be bound with the session ID number; when the system sends data to the outside, the information security gateway receives the data through the communication module, performs integrity authentication on the data, then encrypts the data through a negotiated secret key, and finally transmits the data to the server through the communication module. When the information security management module receives data through the communication module, the data is decrypted first, integrity verification is carried out on the data after decryption, and if verification is passed, the data is sent out through the communication module; otherwise, the data is discarded.

In an embodiment, the identity authentication is realized by adopting an asymmetric key with a signature.

In an embodiment, the signature is realized by a pair of asymmetric keys which are trusted by the information security management module and the system, wherein the private key is used for signing the identities of the two communication parties, and the public key is used for verifying that the identities of the two communication parties are signed. Signing identity comprises a public and a private key pair. In other words, signing identity is referred to as the common name of the certificates which are installed in the user's machine.

In an embodiment, both communication parties need to authenticate their own identities through a pair of asymmetric keys, and a task in charge of communication with the information security management module of the system is identified by a unique pair of asymmetric keys.

In an embodiment, the dynamic negotiation key is encrypted by adopting a Rivest-Shamir-Adleman (RSA) encryption algorithm. RSA is a public key cryptosystem that is widely used for secure data transmission. The negotiated keys include a data encryption key and a data integrity check key.

In an embodiment, the data encryption method is a Triple Data Encryption Algorithm (3DES) encryption algorithm. The integrity check algorithm is a Hash-based Message Authentication Code (HMAC-MD5-128) algorithm. When data is output, the integrity check calculation is carried out on the data, the calculated Message Authentication Code (MAC) value is added with the header of the value data message, then the data (including the MAC of the header) is encrypted by using a 3DES algorithm, the header information of a security layer is added after the data is encrypted, and then the data is sent to the next layer for processing. In an embodiment the next layer refers to a transport layer in the Transmission Control Protocol/Internet Protocol (TCP/IP) model.

The information security management module ensures the safety, reliability, and confidentiality of the communication between the system and the server through the identity authentication when the communication between the two communication parties starts the data encryption and the data integrity authentication. The method is particularly suitable for an embedded platform which has less resources and is not connected with a Public Key Infrastructure (PKI) system and can ensure that the safety of the data on the server cannot be compromised by a hacker attack under the condition of the Internet by ensuring the safety and reliability of the communication between the system and the server

Technical Result: The disclosure provides a unique two-phased solution that provides an individualized patient prediction and prescription for preventing Opioid Use Disorder (OUD). This two-phased solution uses the following methodologies:
a. Predictive analytics for identifying patients who are at risk of developing OUD, and
b. Prescriptive analytics for providing treatment recommendations.
c. Improving clinical trials on OUD patients using predictive analytics.

### Differentiation—Traditional Risk Scoring in the Industry Using Descriptive Analytics:

Over the past decade, several surveys and analytics models have been designed to identify patients who have already developed OUD. These models are geared towards addressing a population that is in immediate need of rehabilitation and is subject to potential overdose. The risk scoring models that are in use today bring value to prescribers and insurers when taking next steps to address the future ramifications of addiction. The models and surveys are descriptive in nature and identify the current state of a patient. A descriptive model is a data analysis method that aims to describe and understand existing patterns or trends within a dataset by summarizing key characteristics, essentially providing an overview of the current state.

In contrast to existing descriptive models that only identify patients who have already developed OUD, the current disclosed technological solution uses predictive analytics to identify patients who carry the risk of developing OUD along with their contributing attributes.

### How the Technical Solution is a Technological Advancement:

The technological advancement in this solution arises from the integration of predictive and prescriptive analytics, combined with Explainable AI (XAI), subscriber and prescriber profiling, and a decision support system (DSS), which is integrated with PDMP and/or EHR systems, for prescribers and pharmacists to improve opioid use disorder (OUD) management.

Predictive analytics employs machine learning models trained on PDMP and EHR data to generate risk scores, estimating a patient's likelihood of developing OUD. These models use advanced frameworks like TensorFlow and PyTorch and operate on CPU, GPU, and Edge computing environments for real-time risk assessment. Prescriptive analytics then translates these predictions into treatment recommendations, such as dosage modifications, alternative medications, craving-suppression therapies, behavioral interventions, and withdrawal management strategies.

A key advancement is the incorporation of Explainable AI (XAI), which attributes factors influencing risk scores, providing transparency in decision-making. This includes identifying patient attributes, prescription history, and prescriber behavior that contribute to OUD risk. Subscriber profiling analyzes prescription frequency, dosage patterns, multi-provider visits, and medication adherence, helping detect overuse or misuse trends. Simultaneously, prescriber profiling assesses prescription patterns, drug preferences, refill tendencies, and deviations from standard medical guidelines, identifying potential overprescription or improper opioid dispensing practices.

To further enhance clinical decision-making, the system integrates a Decision Support System (DSS) for prescribers and pharmacists, enabling real-time alerts, patient-specific recommendations, and compliance monitoring. This facilitates informed treatment decisions, risk mitigation, and proactive interventions to prevent opioid misuse. Additionally, the solution includes feature eStores for reusable data processing, responsible AI governance, and observability tools like Datadog, ensuring continuous model monitoring, auditability, and compliance with healthcare regulations. By automating risk assessment, profiling, and decision support, this solution surpasses traditional rule-based systems, making opioid management more efficient, transparent, and adaptive to evolving patient and prescriber behaviors.

The system is designed to address the above challenges by providing an individualized patient prediction and prescription for preventing opioid use disorder. The system leverages advanced technologies, including machine learning and artificial intelligence, providing an end-to-end solution towards identification and prevention of OUD in patients.

The descriptions of the one or more embodiments are for purposes of illustration but are not exhaustive or limiting to the embodiments described herein. Many modifications and variations will be apparent to those of ordinary skill in the art without departing from the scope and spirit of the embodiments described. The terminology used herein best explains the principles of the embodiments, the practical application and/or technical improvement over technologies found in the marketplace, and/or to enable others of ordinary skill in the art to understand the embodiments described herein.

### INCORPORATION BY REFERENCE

All references, including granted patents and patent application publications, referred herein are incorporated herein by reference in their entirety:
U.S. Patent Publication, U.S. Pat. No. 11,640,857B2, titled, “Techniques for providing referrals for opioid use disorder treatment”.
U.S. Patent Publication, U.S. Pat. No. 10,791,987B2, titled, “Methods and systems for managing a risk of medication dependence”.
U.S. Patent Publication, U.S. Pat. No. 10,998,105B2-Methods and systems for evaluation of risk of substance use disorders.
U.S. Patent Application Publication, US20240087750A1—Machine learning systems and methods for predicting risk of incident opioid use disorder and opioid overdose.
U.S. Patent Application Publication, US20210202104A1—Identifying and measuring patient overdose risk.

### REFERENCES

1. Krebs E, Enns B, Evans E, et al. Cost-Effectiveness of Publicly Funded Treatment of Opioid Use Disorder in California. Ann Intern Med 2018; 168 (1): 10-19. (In eng). DOI: 10.7326/M17-06112664093 [pii].
2. Statistics NCfH. Provisional Drug Overdose Death Counts. Centers for Disease Control and Prevention: 2021. (https://www.cdc.gov/nchs/nvss/vsrr/drug-overdose-data.htm).
3. Services LCHH. CDC: Drug Overdose Deaths in US top 100,000. 2021. (https://www.co.lincoln.or.us/hhs/page/cdcdrug-overdose-deaths-us-top-100000).
4. NIDA. Overdose Death Rates. (https://www.drugabuse.gov/related-topics/trends-statistics/overdose-deathrates).
5. CDC. Prescription Opioid Data. (https://www.cdc.gov/drugoverdose/data/prescribing.html?CDC_AA_refVal=https%3A%2F%2F www.cdc.gov%2Fdrugoverdose%2Fdata%2Foverdose.html).
6. Dasgupta N, Beletsky L, Ciccarone D. Opioid Crisis: No Easy Fix to Its Social and Economic Determinants. American Journal of Public Health 2018; 108 (2): 182-186. DOI: 10.2105/ajph.2017.304187.
7. Goldstick J E, Guy G P, Losby J L, Baldwin G, Myers M, Bohnert A S. Changes in Initial Opioid Prescribing Practices After the 2016 Release of the CDC Guideline for Prescribing Opioids for Chronic Pain. JAMA network open 2021; 4 (7): e2116860-e2116860.
8. Abuse NIOD. Prescription Opioids and Heroin Research Report. 2018. (https://nida.nih.gov/download/19774/prescription-opioids-heroin-researchreport.pdf?utm_medium=email&utm_source=transaction).
9. Lankenau S E, Teti M, Silva K, Bloom J J, Harocopos A, Treese M. Initiation into prescription opioid misuse amongst young injection drug users. International Journal of Drug Policy 2012; 23 (1): 37-44.
10. Altarum. Economic Toll of Opioid Crisis in U.S. Exceeded $1 Trillion Since 2001. (https://altarum.org/about/newsand-events/economic-toll-of-opioid-crisis-in-u-s-exceeded-1-trillion-since-2001).
11. CDC. New data show continuing opioid epidemic in the United States. (https://www.cdc.gov/media/releases/2016/p1216-continuing-opioid-epidemic.html).
12. Cunningham C O. Opioids and HIV infection: from pain management to addiction treatment. Topics in antiviral medicine 2018; 25 (4): 143.
13. Webster L, St Marie B, McCarberg B, Passik S D, Panchal S J, Voth E. Current status and evolving role of abuse deterrent opioids in managing patients with chronic pain. J Opioid Manag 2011; 7 (3): 235-45. (In eng) (http://www.ncbi.nlm.nih.gov/pubmed/21823554).
14. Fitzcharles M A, Ste-Marie P A, Gamsa A, Ware M A, Shir Y. Opioid use, misuse, and abuse in patients labeled as fibromyalgia. Am J Med 2011; 124 (10): 955-60. (In eng). DOI: S0002-9343 (11) 00494-3 [pii] 10.1016/j.amjmed.2011.05.031.
15. Florence C S, Zhou C, Luo F, Xu L. The Economic Burden of Prescription Opioid Overdose, Abuse, and Dependence in the United States, 2013. Med Care 2016; 54 (10): 901-6. (In eng). DOI: 10.1097/MLR.000000000000062500005650-201610000-00002 [pii].
16. Control CfD. Drug Overdose Deaths in the U.S. Top 100,000 Annually. 2021. (https://www.cdc.gov/nchs/pressroom/nchs_press_releases/2021/20211117.htm).
17. Guy G P, Jr., Zhang K, Bohm M K, et al. Vital Signs: Changes in Opioid Prescribing in the United States, 2006-2015. MMWR Morb Mortal Wkly Rep 2017; 66 (26): 697-704. (In eng). DOI: 10.15585/mmwr.mm6626a4.
18. Unick G, Ciccarone D. U S Regional and Demographic Differences in Prescription Opioid and Heroin-Related Overdose Hospitalizations. The International journal on drug policy 2017; 46:112-119. DOI: 10.1016/j.drugpo.2017.06.003.
19. Barnett M L, Olenksi A R, Jena A B. Opioid Prescribing by Emergency Physicians and Risk of Long-Term Use: N Engl J Med. 2017 May 11; 376 (19): 1896. doi: 10.1056/NEJMc1703338.
20. Chisholm-Burns M A, Spivey C A, Sherwin E, Wheeler J, Hohmeier K. The opioid crisis: origins, trends, policies, and the roles of pharmacists. American journal of health-system pharmacy 2019; 76 (7): 424-435.
21. James L. Attorney General James Secures Over $10 Billion from CVS and Walgreens for Communities Nationwide to Combat the Opioid Crisis. 2022.
22. Schuler M S, Schell T L, Wong E C. Racial/ethnic differences in prescription opioid misuse and heroin use among a national sample, 1999 2018. Drug and alcohol dependence 2021; 221:108588.
23. Furr-Holden D, Milam A, Wang L, Sadler R. African Americans now outpace whites in opioid-involved overdose deaths: a comparison of temporal trends from 1999 to 2018. Addiction [internet] 2020 [cited 2020 Sep. 25].
24. Wu L-T, Zhu H, Swartz M S. Treatment utilization among persons with opioid use disorder in the United States. Drug and Alcohol Dependence 2016; 169:117-127. DOI: 10.1016/j.drugalcdep.2016.10.015.
25. Legislatures NCoS. Prescribing Policies: States Confront Opioid Overdose Epidemic. (http://www.ncsl.org/research/health/prescribing-policies-states-confront-opioid-overdose-epidemic.aspx).
26. Soelberg C D, Brown R E, Jr., Du Vivier D, Meyer J E, Ramachandran B K. The US Opioid Crisis: Current Federal and State Legal Issues. Anesth Analg 2017; 125 (5): 1675-1681.
27. State Prescription Drug Monitoring Programs. Office of Diversion Control; U.S. Department of Justice Drug Enforcement Administration. (http://www.deadiversion.usdoj.gov/faq/rx_monitor.htm#1).
28. Haffajee R L, Jena A B, Weiner S G. Mandatory Use of Prescription Drug Monitoring Programs. JAMA 2015; 313 (9): 891-892. DOI: 10.1001/jama.2014.18514.
29. Barlas S. U.S. and States Ramp Up Response to Opioid Crisis: Regulatory, Legislative, and Legal Tools Brought to Bear. Pharmacy and Therapeutics 2017; 42 (9): 569-592. (http://www.ncbi.nlm.nih.gov/pmc/articles/PMC5565130/).
30. Medicine ASoA. Public Policy Statement on Patient Review and Restriction (PRR) Programs. (https://www.asam.org/advocacy/find-a-policy-statement/view-policy-statement/public-policystatements/2016/05/10/patient-review-and-restriction-(prr)-programs).
31. Roberts AWea. Controlled Substance Lock-In Programs: Examining an Unintended Consequence of a Prescription Drug Abuse Policy. Health Affairs 2016; 35 (10) (https://www.healthaffairs.org/doi/10.1377/hlthaff.2016.0355).
32. Brennan T, Creager R., Polinski, J. M. Pharmacy Benefit Management of Opioid Prescribing: The Role of Employers and Insurers. Health affairs (Project Hope) 2017 (https://www.healthaffairs.org/do/10.1377/hblog20170921.062092/full/).
33. Appold K. Will PBMs Be the Next Target of Opioid Lawsuits? Managed Healthcare Executive. (http://www.managedhealthcareexecutive.com/business-strategy/will-pbms-be-next-target-opioid-lawsuits).
34. Joseph A. Supreme Court rules that doctors need to knowingly mis prescribe opioids for it to be a crime. Stat 2022 (https://www.statnews.com/2022/06/27/supreme-court-rules-that-doctors-need-to-knowingly-misprescribeopioids-for-it-to-be-a-crime/).
35. Cunningham C, Edlund M J, Fishman M, et al. The ASAM national practice guideline for the treatment of opioid use disorder: 2020 focused update. J Addict Med 2020; 14 (2S Suppl 1): 1-91.
36. Kashyap A, Callison-Burch C, Boland M R. A deep learning method to detect opioid prescription and opioid use disorder from electronic health records. Int J Med Inform. 2023 March;171:104979. doi: 10.1016/j.ijmedinf.2022.104979. Epub 2022 Dec. 31. PMID: 36621078; PMCID: PMC9898169.
37. Dong X, Rashidian S, Wang Y, Hajagos J, Zhao X, Rosenthal R N, Kong J, Saltz M, Saltz J, Wang F. Machine Learning Based Opioid Overdose Prediction Using Electronic Health Records. AMIA Annu Symp Proc. 2020 Mar. 4; 2019:389-398. PMID: 32308832; PMCID: PMC7153049.
38. Walid F. Gellad, Qingnan Yang, Kayleigh M. Adamson, Courtney C. Kuza, Jeanine M. Buchanich, Ashley L. Bolton, Stanley M. Murzynski, Carrie Thomas Goetz, Terri Washington, Michael F. Lann, Chung-Chou H. Chang, Katie J. Suda, Lu Tang, Development and validation of an overdose risk prediction tool using prescription drug monitoring program data, Drug and Alcohol Dependence, Volume 246, 2023, 109856, ISSN 0376-8716, https://doi.org/10.1016/j.drugalcdep.2023.109856.

1-43. (canceled)

44. A system comprising:
a processor storing instructions in a non-transitory memory that, when executed, cause the processor to:
receive, input data of a patient, wherein the input data comprises one or more of a first patient data, a first prescription data, a first drug data, a first dispenser data, and a first prescriber data;
derive, one or more attribute variables, based on the input data, wherein the attribute variables comprise one or more of a second patient data, a second prescription data, a second drug data, a second dispenser data, and a second prescriber data;
predict, using a predictive analytics module comprising a first artificial intelligence and machine learning model, a risk score for the patient based on the attribute variables, wherein the risk score is a probability of the patient developing an opioid use disorder, wherein the first artificial intelligence and machine learning model that provides explainable outcome and is pre-trained on a training database; and
determine, a list of the attribute variables that contribute to the risk score, wherein the list comprises a percentage contribution of each of the attribute variables in the list to the risk score, wherein the list comprises a subset of the attribute variables;
provide, using a prescriptive analytics module comprising a second artificial intelligence and machine learning model, a treatment recommendation based on the risk score and the attribute variables, wherein the second artificial intelligence and machine learning model is pre-trained on the training database, wherein the treatment recommendation comprises one or more of a dosage, an alternate medication, a craving-suppression medication, a behavioral intervention, a therapy, a withdrawal reduction treatment, and educational materials to overcome the opioid use disorder; and
wherein the system is configured to one or more of identifying and preventing the opioid use disorder in the patient, wherein the patient is being treated for pain with a prescription drug, and wherein the prescription drug is an opioid.

45. The system of claim 44, wherein the input data is considered from one or more databases, wherein the databases comprise Prescription Drug Monitoring Program (PDMP) and Electronic Health Record (EHR).

46. The system of claim 44, wherein the first patient data comprises at least one or more of patient identification details, patient date of birth, and patient location.

47. The system of claim 44, wherein the first prescription data comprises at least one or more of prescription filled data, prescription date, and prescription number.

48. The system of claim 44, wherein the first drug data comprises at least one or more of drug name, drug strength, drug form, drug quantity, and number of days of supply.

49. The system of claim 44, wherein the first dispenser data comprises one or more of dispenser identification details and dispenser location.

50. The system of claim 44, wherein the first prescriber data comprises one or more of prescriber identification details and prescriber location.

51. The system of claim 44, wherein the second patient data comprises one or more of patient unique identification, patient age, and patient location.

52. The system of claim 44, wherein the second prescription data comprises one or more of number of prescriptions in a time period, days since last prescription, and average duration between prescriptions.

53. The system of claim 44, wherein the second drug data comprises one or more of average drug quantity, highest drug quantity, average days of supply, highest days of supply, highest drug strength, lowest drug strength as per the first prescription data and the first drug data.

54. The system of claim 44, wherein the second dispenser data comprises one or more of dispenser identification details, dispenser location, highest number of prescriptions filled by a dispenser, identification details of most frequently used dispenser for filling the prescription drug.

55. The system of claim 44, wherein the second prescriber data comprises one or more of prescriber identification details, prescriber location, highest number of prescriptions prescribed, and most frequent prescriber identification details.

56. The system of claim 44, wherein the first artificial intelligence and machine learning model comprises a logistic regression model configured to explain why a patient is at risk.

57. The system of claim 56, wherein the logistic regression model applies a linear and weighted contribution of the input data; and wherein the logistic regression model comprises a multivariate logistic regression.

58. The system of claim 44, wherein the system is configured for use by a physician as a decision support system for prescribing one or more of the prescription drug and the treatment recommendation.

59. The system of claim 44, wherein the second artificial intelligence and machine learning model comprises a clustering model, wherein the clustering model comprises one of recursive partitioning and random forest clustering.

60. A method comprising,
receiving, input data of a patient, wherein the input data comprises one or more of a first patient data, a first prescription data, a first drug data, a first dispenser data, and a first prescriber data;
deriving, one or more attribute variables, based on the input data, wherein the attribute variables comprise one or more of a second patient data, a second prescription data, a second drug data, a second dispenser data, and a second prescriber data;
predicting, using a predictive analytics module comprising a first artificial intelligence and machine learning model, a risk score for the patient based on the attribute variables, wherein the risk score is a probability of the patient developing an opioid use disorder, wherein the first artificial intelligence and machine learning model is pre-trained on a training database; and
determining, a list of the attribute variables that contribute to the risk score, wherein the list comprises a percentage contribution of each of the attribute variables in the list to the risk score, wherein the list comprises a subset of the attribute variables;
providing, using a prescriptive analytics module comprising a second artificial intelligence and machine learning model, a treatment recommendation based on the risk score and the attribute variables, wherein the second artificial intelligence and machine learning model is pre-trained on the training database, wherein the treatment recommendation comprises one or more of a dosage, an alternate medication, and craving-suppression medication, a behavioral intervention, a therapy, a withdrawal reduction treatment, and educational materials to overcome the opioid use disorder; and
wherein the method is configured to one or more of identifying and preventing the opioid use disorder in the patient, wherein the patient is being treated for pain with a prescription drug, and wherein the prescription drug is an opioid.

61. The method of claim 60, wherein the risk score is a value between 0 and 1; and wherein the risk score along with the list of attributes is one or more of displayed on a display, stored to a database, and generates an alarm.

62. The method of claim 60, wherein the first artificial intelligence and machine learning model and the second artificial intelligence and machine learning model are retrained based on false positive and false negative results during a period of use of the first artificial intelligence and machine learning model and the second artificial intelligence and machine learning model.

63. A non-transitory computer-readable medium having stored thereon instructions executable by a computer system to perform operations comprising:
receiving, input data of a patient, wherein the input data comprises one or more of a first patient data, a first prescription data, a first drug data, a first dispenser data, and a first prescriber data;
deriving, one or more attribute variables, based on the input data, wherein the attribute variables comprise one or more of a second patient data, a second prescription data, a second drug data, a second dispenser data, and a second prescriber data;
predicting, using a predictive analytics module comprising a first artificial intelligence and machine learning model, a risk score for the patient based on the attribute variables, wherein the risk score is a probability of the patient developing an opioid use disorder, wherein the first artificial intelligence and machine learning model is pre-trained on a training database; and
determining, a list of the attribute variables that contribute to the risk score, wherein the list comprises a percentage contribution of each of the attribute variables in the list to the risk score, wherein the list comprises a subset of the attribute variables;
providing, using a prescriptive analytics module comprising a second artificial intelligence and machine learning model, a treatment recommendation based on the risk score and the attribute variables, wherein the second artificial intelligence and machine learning model is pre-trained on the training database, wherein the treatment recommendation comprises one or more of a dosage, an alternate medication, and craving-suppression medication, a behavioral intervention, a therapy, a withdrawal reduction treatment, and educational materials to overcome the opioid use disorder; and
wherein the operations are configured to one or more of identifying and preventing the opioid use disorder in the patient, wherein the patient is being treated for pain with a prescription drug, and wherein the prescription drug is an opioid.
